(19) United States
(12) Patent Application Publication
(10) Pub. No.: US 2023/0338569 A1
(43) Pub. Date: Oct. 26, 2023

(54) NaPi2b-TARGETED POLYMER ANTIBODY-DRUG CONJUGATE FOR THE TREATMENT OF OVARIAN CANCER

(71) Applicant: Mersana Therapeutics, Inc., Cambridge, MA (US)

(72) Inventor: Robert A. BURGER, Jenkintown, PA (US)

(21) Appl. No.: 17/930,924

(22) Filed: Sep. 9, 2022

Related U.S. Application Data
(60) Provisional application No. 63/304,185, filed on Jan. 28, 2022.
(60) Provisional application No. 63/242,319, filed on Sep. 9, 2021.

(51) Int. Cl. A61K 47/68; C07K 16/30

(52) U.S. Cl. CPC A61K 47/6869; C07K 16/3069; A61K 47/68031

(57) ABSTRACT

Disclosed herein are dosing regimens for a NaPi2b-targeted antibody-drug conjugates for treating recurrent, platinum-sensitive ovarian cancer.

### CROSS-REFERENCE TO RELATED APPLICATIONS

This application claims priority to, and the benefit of, U.S. Provisional Application No. 63/242,319 filed Sep. 9, 2021, and U.S. Provisional Application No. 63/304,185 filed Jan. 28, 2022. The contents of each of these applications are hereby incorporated by reference in their entireties.

### INCORPORATION BY REFERENCE OF SEQUENCE LISTING

The Sequence Listing XML associated with this application is provided electronically in txt file format and is hereby incorporated by reference into the specification. The name of the txt file containing the Sequence Listing is MRSN_036_001US_SeqList_ST26.xml. The xml file is 16.2 KB, created on Sep. 6, 2022, and is being submitted electronically via USPTO Patent Center.

### FIELD OF THE INVENTION

This disclosure relates generally to dosing regimens for administering a NaPi2b-targeted polymer antibody-drug conjugates for the treatment of ovarian cancer.

### BACKGROUND OF THE INVENTION

NaPi2b (SLC34A2, NaPiIIb, Npt2), a multi-transmembrane, sodium-dependent phosphate transporter (Xu et al. Genomics 62:281-284 (1999)), is normally expressed at the brush border membrane of mammalian small intestine and participates in the transcellular inorganic phosphate (Pi) absorption, contributing to the maintenance of phosphate homeostasis in the body. The expression of NaPi2b at the protein level has been detected in the liver, at the apical surface of epithelial cells of mammary, salivary glands, and bronchi, and in the lungs, testis, thyroid gland, small intestine, and uterus. Mutations in NaPi2b have been associated with clinical syndromes of alveolar and testicular micro lithiasis. NaPi2b is highly expressed in non-squamous non-small cell lung cancer (NSCLC), non-mucinous ovarian cancer and papillary thyroid cancer. NaPi2b-positive tissue immunoreactivity is present in 61% of NSCLC, and 92% ovarian cancer specimens.

Ovarian cancer is one of the most common gynecologic malignancies and the fifth most frequent cause of cancer death in women. The high mortality rate results in part from the frequent diagnosis of ovarian cancer at advanced stages and the mortality rate is approximately 65% of the incidence rate. The constellation of diseases commonly referred to as “ovarian cancer,” includes epithelial ovarian, primary peritoneal and fallopian tube carcinomas and represents the most common cause of gynecologic cancer death in the United States. The lethality of this disease has been attributed largely to advanced stage at diagnosis (and absence of effective screening for potentially early-stage disease). In addition, after standard management of newly diagnosed advanced ovarian cancer (including surgical cytoreduction and platinum/taxane chemotherapy with or without the anti-VEGF monoclonal antibody bevacizumab, and with or without poly ADP-ribose polymerase (PARP) inhibitors (PARPi)), the vast majority of patients will experience recurrence and die of disease. The benefits of standard therapies are limited by both intrinsic and acquired drug resistance. Thus, there is a need for the development of new agents with activity against ovarian cancer, including those that target the biological activities of NaPi2b.

### SUMMARY OF THE INVENTION

The disclosure provides a method of treating an ovarian cancer (including fallopian tube and primary peritoneal cancer) in a subject having a recurrent, platinum-sensitive ovarian cancer, comprising administering to the subject a NaPi2b-targeted antibody polymer-drug conjugate (XMT-1536) by infusion at a dose of between 20 mg/m² to 36 mg/m² on the first day of treatment and every four weeks thereafter, wherein the NaPi2b-targeted antibody polymer-drug conjugate is:

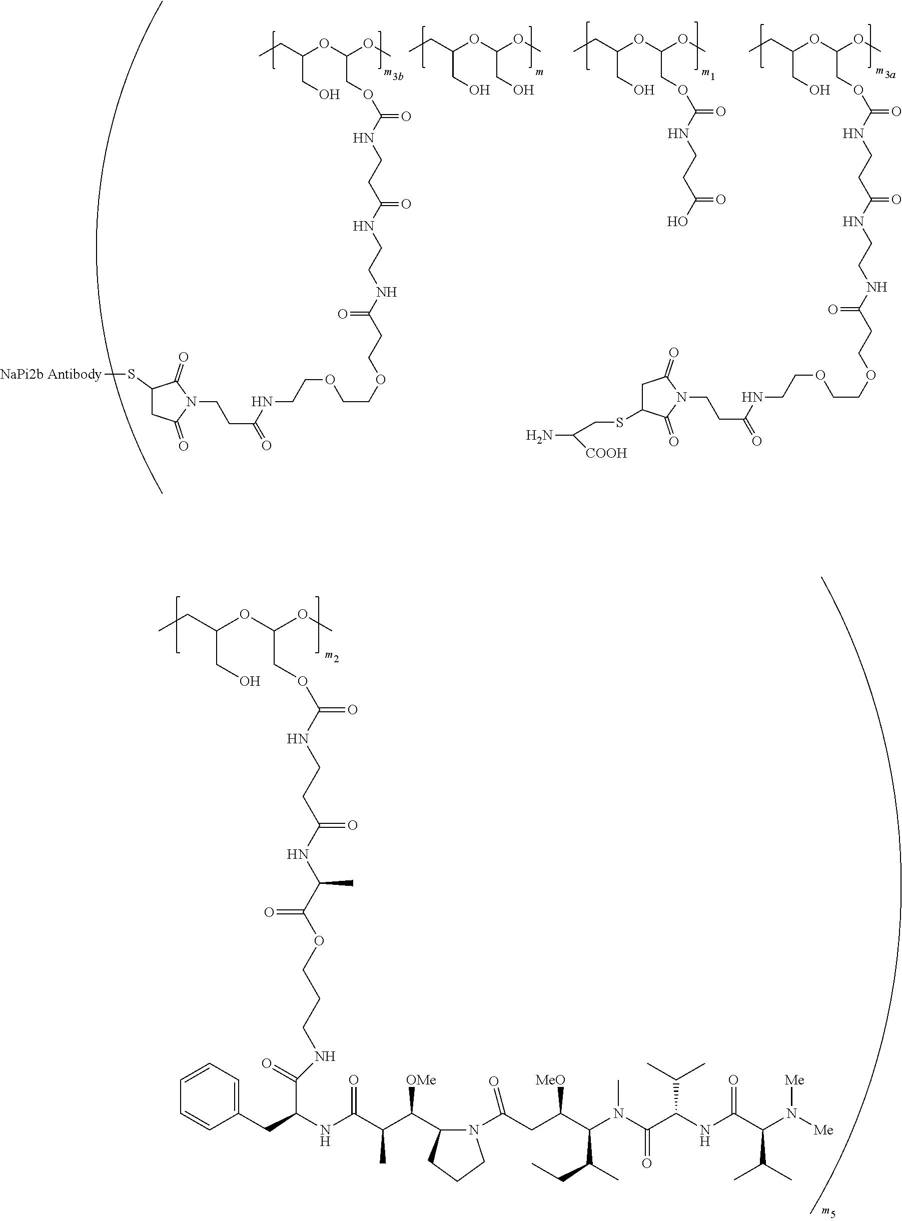

wherein: the polymer-drug conjugate comprises a polymeric scaffold comprising poly(1-hydroxymethylethylene hydroxymethyl-formal) (PHF), wherein the PHF has a molecular weight ranging from 5 kDa to 10 kDa; m is an integer from 20 to 75, m₁ is an integer from about 5 to about 35, m₂ is an integer from about 3 to about 10, m₃ₐ is an integer from 0 to about 4, m_{3b} is an integer from 1 to about 5, the sum of m, m₁, m₂, m₃ₐ, and m_{3b} ranges from about 40 to about 75, m₅ is an integer from about 2 to about 6, the NaPi2b-targeted antibody (XMT-1535) comprises a variable light chain complementarity determining region 1 (CDRL1) comprising the amino acid sequence SASQDIGNFLN (SEQ ID NO: 8); a variable light chain complementarity determining region 2 (CDRL2) comprising the amino acid sequence YTSSLYS (SEQ ID NO: 9); a variable light chain complementarity determining region 3 (CDRL3) comprising the amino acid sequence QQYSKLPLT (SEQ ID NO: 10); a variable heavy chain complementarity determining region 1 (CDRH1) comprising the amino acid sequence GYTFTGYNIH (SEQ ID NO: 5); a variable heavy chain complementarity determining region 2 (CDRH2) comprising the amino acid sequence AIYPGNGDTSYKQKFRG (SEQ ID NO: 6); and a variable heavy chain complementarity determining region 3 (CDRH3) comprising the amino acid sequence GETARATFAY (SEQ ID NO: 7).

In some embodiments, XMT-1535 comprises a variable heavy chain comprising the amino acid sequence of SEQ ID NO: 3 and a variable light chain comprising the amino acid sequence of SEQ ID NO: 4.

In some embodiments, XMT-1535 comprises a heavy chain comprising the amino acid sequence of SEQ ID NO: 1 and a light chain comprising the amino acid sequence of SEQ ID NO: 2.

In some embodiments, PHF has a molecular weight ranging from about 5 kDa to about 10 kDa, m is an integer from 30 to about 35, m₁ is an integer from 8 to about 10, m₂ is an integer from 2 to about 5, m₃ₐ is an integer from 0 to about 1, m_{3b} is an integer from 1 to about 2, the sum of m₃ₐ and m_{3b} ranges from 1 and about 4, and m₅ is an integer from about 2 to about 6. In some embodiments, the ratio between m₂ and XMT-1535 is about 16:1 to 10:1. In some embodiments, the ratio between m₂ and XMT-1535 is about 12:1 to 8:1. In some embodiments, the ratio between m₂ and XMT-1535 is about 10:1 to 8:1. In some embodiments, the ratio between m₂ and XMT-1535 is about 10:1.

In some embodiments, m₅ is an integer from about 2 to about 5. In some embodiments, m₅ is an integer from about 2 to about 4. In some embodiments, m₅ is an integer from about 3 to about 4.

In some embodiments, the subject is administered the NaPi2b-targeted antibody polymer-drug conjugate by IV infusion at a dose of 20 mg/m², 25 mg/m², 30 mg/m², 36 mg/m² or 43 mg/m² once every 4 weeks. In some embodiments, the conjugate dose is 20 mg/m². In some embodiments, the conjugate dose is 25 mg/m². In some embodiments, the conjugate dose is 30 mg/m². In some embodiments, the conjugate dose is 36 mg/m². In some embodiments, the conjugate dose is 43 mg/m². In some embodiments, the conjugate dose is 36 mg/m² with BSA capped at 2.2 m². In some embodiments, the conjugate dose is 30 mg/m² with BSA capped at 2.2 m². In some embodiments, the conjugate dose is 25 mg/m² with BSA capped at 2.2 m². In some embodiments, the conjugate dose is 43 mg/m² with BSA capped at 1.8 m². In some embodiments, the conjugate dose is 36 mg/m² up to a maximum of approximately 80 mg. In some embodiments, the conjugate dose is 30 mg/m² up to a maximum of approximately 80 mg. In some embodiments, the conjugate dose is 25 mg/m² up to a maximum of approximately 80 mg. In some embodiments, the conjugate dose is about 80 mg.

In some embodiments, the NaPi2b-targeted antibody polymer-drug conjugate is administered for a maximum of 18 months or until disease progression, unacceptable toxicity, voluntary discontinuation, or death, whichever comes first.

In some embodiments, the ovarian cancer is high-grade serous ovarian cancer. In some embodiments, the subject has a recurrent, platinum-sensitive HGSOC (including fallopian tube and primary peritoneal cancer) and has received up to 4 prior lines of systemic therapy and is currently in response to their treatment.

In some embodiments, the subject has a recurrent, platinum-sensitive HGSOC (including fallopian tube and primary peritoneal cancer) and has received treatment with 4 to 8 cycles of platinum-based combination chemotherapy in the 2nd-4th line setting for the treatment of platinum-sensitive recurrent disease, with no evidence of disease (NED)/complete response (CR)/partial response (PR)/ or stable disease (SD) as best response. Platinum-based combination chemotherapy includes, but is not limited to, carboplatin or cisplatin±paclitaxel, docetaxel, pegylated liposomal doxorubicin or gemcitabine.

In some embodiments, the subject who has received only one prior line of platinum-based therapy or who has received more than 4 prior lines of platinum-based therapy is excluded from receiving the NaPi2b-targeted antibody polymer-drug conjugate by IV infusion at a dose of 20 mg/m², 25 mg/m², 30 mg/m², 36 mg/m² or 43 mg/m² once every 4 weeks.

In some embodiments, the subject who has received bevacizumab in combination with their most recent platinum-based regimen is excluded from receiving the NaPi2b-targeted antibody polymer-drug conjugate by IV infusion at a dose of 20 mg/m², 25 mg/m², 30 mg/m², 36 mg/m² or 43 mg/m² once every 4 weeks.

In some embodiments, the subject has a tumor positive for NaPi2b, as measured by central laboratory (archived or recent biopsy). In some embodiments, the subject has a Tumor Proportion Scoring (TPS) of ≥75.

In some embodiments, the subject experiences reduced and/or no progression of the ovarian cancer following treatment with the NaPi2b-targeted antibody polymer-drug conjugate relative to treatment with placebo.

In some embodiments, the subject experiences improved progression-free survival following treatment with the NaPi2b-targeted antibody polymer-drug conjugate relative to treatment with placebo.

Other features and advantages of the invention will be apparent from the following detailed description and claims.

### DETAILED DESCRIPTION

The present disclosure provides methods of treating recurrent, platinum-sensitive ovarian cancer, by administration of a NaPi2b-targeted polymer antibody-drug conjugate (XMT-1536) that specifically bind to the extracellular region of SLC34A2. In some aspects, the ovarian cancer is high-grade serous ovarian cancer (HGSOC). Specifically, the invention provides dosing regimens for the treatment of NaPi2b expressing ovarian cancers by administration as an intravenous infusion. XMT-1536 is comprised of about 8-12 molecules of auristatin F-hydroxypropyl amide (AF HPA) conjugated to a cysteine moiety of a NaPi2b monoclonal antibody (XMT-1535) via a poly(1-hydroxymethylethylene hydroxymethyl-formal) (PHF) scaffold.

Patients with recurrent, platinum-sensitive ovarian cancer, including HGSOC (also including fallopian tube and primary peritoneal cancer) and who have received treatment with 4 to 8 cycles of platinum-based combination chemotherapy in the 2nd-4th line setting for the treatment of platinum-sensitive recurrent disease, with no evidence of disease (NED)/complete response (CR)/partial response (PR)/ or stable disease (SD) as best response are intravenously administered XMT-1536 once every 4 weeks. Accordingly, the invention features methods of treating recurrent, platinum-sensitive ovarian cancer, including HGSOC, by administering to a subject, i.e., human, in a dose escalation study an infusion dose of XMT-1536 at 20 mg/m², 25 mg/m², 30 mg/m², 36 mg/m² each capped at a BSA of 2.2 m² or 43 mg/m² capped at a BSA of 1.8 m². The subject is administered an infusion dose of XMT-1536 for a maximum of 18 months or until disease progression, unacceptable toxicity, voluntary discontinuation, or death, whichever comes first.

In some embodiments, platinum-based combination chemotherapy includes, but is not limited to, carboplatin or cisplatin with or without paclitaxel, docetaxel, pegylated liposomal doxorubicin or gemcitabine.

In some embodiments the subject has been identified as having NaPi2b expression. In some embodiments, the NaPi2b expression is in the form of a NaPi2b expressing tumor. NaPi2b expression is detected by methods known in the art. For example, by immunohistochemistry (IHC) analysis, fluorescent in situ hybridization (FISH) assay or RNA expression analysis of NaPi2b transcript or other genes related to cancer measured in tumor samples. Blood-based biomarkers, which may include serum cytokines, circulating immune cells, and circulating tumor cells can also be used to determine the NaPi2b expression levels.

### NaPi2b Antibodies

The NaPi2b antibodies suitable for the methods of the disclosure specifically bind to the extracellular region of SLC34A2. The disclosure further provides NaPi2b-targeted monoclonal antibodies that specifically recognizes NaPi2b, also known as sodium-dependent phosphate transport protein 2B. The NaPi2b antibodies used in the conjugates disclosed herein are capable of and useful in modulating, e.g., blocking, inhibiting, reducing, antagonizing, neutralizing or otherwise interfering with at least one biological activity of NaPi2b. Antibodies disclosed herein also include antibodies that bind soluble NaPi2b. The NaPi2b antibodies specifically bind to an epitope on an extracellular domain (ECD) of the human NaPi2b. These antibodies are collectively referred to herein as “NaPi2b” antibodies.

The NaPi2b antibody-drug conjugates provided herein include antibodies that bind to a NaPi2b epitope with an equilibrium dissociation constant (K_{D} or K_{D}) of ≤1 μM, e.g., ≤100 nM, preferably ≤10 nM, and more preferably ≤1 nM. For example, the NaPi2b antibodies used in the antibody-drug conjugates disclosed herein exhibit a K_{d} in the range approximately between ≤1 nM to about 1 pM.

The NaPi2b antibody-drug conjugates provided herein can include antibodies that serve to modulate, block, inhibit, reduce, antagonize, neutralize or otherwise interfere with the functional activity of NaPi2b. Functional activities of NaPi2b include for example, participating in the transcellular inorganic phosphate (Pi) absorption, thereby contributing to the maintenance of phosphate homeostasis in the body. For example, the NaPi2b antibodies completely or partially inhibit NaPi2b functional activity by partially or completely modulating, blocking, inhibiting, reducing antagonizing, neutralizing, or otherwise interfering with transcellular inorganic phosphate absorption. Transcellular inorganic phosphate absorption activity is assessed using any art-recognized method for detecting transcellular inorganic phosphate absorption activity, including, but not limited to detecting levels of transcellular inorganic phosphate absorption in the presence and absence of an anti-NaPi2b antibody disclosed herein.

The NaPi2b antibodies are considered to completely modulate, block, inhibit, reduce, antagonize, neutralize or otherwise interfere with NaPi2b functional activity when the level of NaPi2b functional activity in the presence of the NaPi2b antibody is decreased by at least 95%, e.g., by 96%, 97%, 98%, 99% or 100% as compared to the level of NaPi2b functional activity in the absence of binding with a NaPi2b antibody described herein. The NaPi2b antibodies are considered to partially modulate, block, inhibit, reduce, antagonize, neutralize or otherwise interfere with NaPi2b functional activity when the level of NaPi2b activity in the presence of the NaPi2b antibody is decreased by less than 95%, e.g., 10%, 20%, 25%, 30%, 40%, 50%, 60%, 75%, 80%, 85% or 90% as compared to the level of NaPi2b activity in the absence of binding with a NaPi2b antibody described herein.

Exemplary antibodies disclosed herein include, the XMT-1535 antibody. These antibodies show specificity for human NaPi2b and they have been shown to inhibit NaPi2b activity.

NaPi2b human or humanized monoclonal antibody, XMT-1535, includes a heavy chain (HC), heavy chain variable region (VH), light chain (LC), and a light chain variable region (VL), as shown in the amino acid and corresponding nucleic acid sequences presented below. The variable heavy chain region and variable light chain region for each antibody are shaded in the amino acid sequences below. The complementarity determining regions (CDRs) of the heavy chain and the light chain are underlined in the amino acid sequences presented below. The amino acids encompassing the complementarity determining regions (CDRs) for the XMT-1535 antibody are as defined by E. A. Kabat et al. (See Kabat, E. A., et al., Sequences of Protein of immunological interest, Fifth Edition, US Department of Health and Human Services, US Government Printing Office (1991)) and are disclosed in U.S. Pat. No. 8,603,474.

>XMT-1535 Heavy Chain Amino Acid Sequence
(Heavy chain variable region (SEQ ID NO: 3)
(Italicized) + IgG1 Heavy chain constant
region (SEQ ID NO: 11))
(SEQ ID NO: 1)
QVQLVQSGAEVVKPGASVKMSCKASGYTFTGYNIHWVKQA
PGQGLEWIGAIYPGNGDTSYKQKFRGRATLTADTSTSTVY
MELSSLRSEDSAVYYCARGETARATFAYWGQGTLVTVSSG
ASTKGPSVFPLAPSSKSTSGGTAALGCLVKDYFPEPVTVS
WNSGALTSGVHTFPAVLQSSGLYSLSSVVTVPSSSLGTQT
YICNVNHKPSNTKVDKRVEPKSCDKTHTCPPCPAPELLGG
PSVFLFPPKPKDTLMISRTPEVTCVVVDVSHEDPEVKFNW
YVDGVEVHNAKTKPREEQYNSTYRVVSVLTVLHQDWLNGK
EYKCKVSNKALPAPIEKTISKAKGQPREPQVYTLPPSREE
MTKNQVSLTCLVKGFYPSDIAVEWESNGQPENNYKTTPPV
LDSDGSFFLYSKLTVDKSRWQQGNVFSCSVMHEALHNHYT
QKSLSLSPG*
CDRH1:
(SEQ ID NO: 5)
GYTFTGYNIH
CDRH2:
(SEQ ID NO: 6)
AIYPGNGDTSYKQKFRG
CDRH3:
(SEQ ID NO: 7)
GETARATFAY
>XMT-1535 Heavy chain variable region nucleic
acid sequence
(SEQ ID NO: 13)
CAAGTTCAGCTGGTTCAGTCTGGCGCCGAGGTTGTGAAAC
CTGGCGCCTCTGTGAAGATGAGCTGCAAGGCCAGCGGCTA
CACCTTCACCGGCTACAACATCCACTGGGTCAAGCAGGCC
CCTGGACAGGGACTCGAATGGATCGGAGCCATCTATCCCG
GCAACGGCGACACCAGCTACAAGCAGAAGTTCCGGGGCAG
AGCCACACTGACCGCCGATACAAGCACCAGCACCGTGTAC
ATGGAACTGAGCAGCCTGAGAAGCGAGGACAGCGCCGTGT
ACTATTGCGCCAGAGGCGAAACAGCCAGAGCCACCTTTGC
CTATTGGGGCCAGGGAACCCTGGTCACCGTTAGCTCT
>XMT-1535 Light Chain Amino Acid Sequence
(Light chain variable region (SEQ ID NO: 4)
(Italicized) + Light chain constant region
(SEQ ID NO: 12))
(SEQ ID NO: 2)
DIQMTQSPSSLSASVGDRVTITCSASQDIGNFLNWYQQKP
GKTVKVLIYYTSSLYSGVPSRFSGSGSGTDYTLTISSLQP
EDFATYYCQQYSKLPLTFGQGTKLELKRRTVAAPSVFIFP
PSDEQLKSGTASVVCLLNNFYPREAKVQWKVDNALQSGNS
QESVTEQDSKDSTYSLSSTLTLSKADYEKHKVYACEVTHQ
GLSSPVTKSFNRGEC
CDRL1:
(SEQ ID NO: 8)
SASQDIGNFLN
CDRL2:
(SEQ ID NO: 9)
YTSSLYS
CDRL3:
(SEQ ID NO: 10)
QQYSKLPLT
>XMT-1535 Light chain variable region nucleic
acid sequence
(SEQ ID NO: 14)
GATATTCAGATGACACAGAGCCCCAGCAGCCTGTCTGCCT
CTGTGGGAGACAGAGTGACCATCACCTGTAGCGCCAGCCA
GGATATCGGCAACTTCCTGAACTGGTATCAGCAGAAACCC
GGCAAGACCGTGAAGGTGCTGATCTACTACACCTCCAGCC
TGTACAGCGGCGTGCCCAGCAGATTTTCTGGCAGCGGCTC
TGGCACCGACTACACCCTGACCATATCTAGCCTGCAGCCT
GAGGACTTCGCCACCTACTACTGCCAGCAGTACAGCAAGC
TGCCCCTGACATTTGGCCAGGGCACCAAGCTGGAACTGAA
G

Also included in the disclosure are antibodies that in to the same epitope or cross compete for binding to the same epitope as the antibodies described herein. For example, antibodies disclosed herein specifically bind to NaPi2b, wherein the antibody binds to an epitope that includes one or more amino acid residues on human NaPi2b (e.g., GenBank Accession No. 095436.3).

Antibodies disclosed herein specifically bind to an epitope on the full-length human NaPi2b comprising the amino acid sequence:

```
(SEQ ID NO: 15)
    1 MAPWPELGDA QPNPDKYLEG AAGQQPTAPD
        KSKETNKTDN TEAPVTKIEL
  51 LPSYSTATLI DEPTEVDDPW NLPTLQDSGI
        KWSERDTKGK ILCFFQGIGR
101 LILLLGFLYF FVCSLDILSS AFQLVGGKMA
        GQFFSNSSIM SNPLLGLVIG
151 VLVTVLVQSS STSTSIVVSM VSSSLLTVRA
        AIPIIMGANI GTSITNTIVA
201 LMQVGDRSEF RRAFAGATVH DFFNWLSVLV
        LLPVEVATHY LEIITQLIVE
251 SFHFKNGEDA PDLLKVITKP FTKLIVQLDK
        KVISQIAMND EKAKNKSLVK
301 IWCKTFTNKT QINVTVPSTA NCTSPSLCWT
        DGIQNWTMKN VTYKENIAKC
351 QHIFVNFHLP DLAVGTILLI LSLLVLCGCL
        IMIVKILGSV LKGQVATVIK
401 KTINTDFPFP FAWLTGYLAI LVGAGMTFIV
        QSSSVFTSAL TPLIGIGVIT
451 IERAYPLTLG SNIGTTTTAI LAALASPGNA
        LRSSLQIALC HFFFNISGIL
501 LWYPIPFTRL PIRMAKGLGN ISAKYRWFAV
        FYLIIFFFLI PLTVFGLSLA
551 GWRVLVGVGV PVVFIIILVL CLRLLQSRCP
        RVLPKKLQNW NFLPLWMRSL
601 KPWDAVVSKF TGCFQMRCCC CCRVCCRACC
        LLCDCPKCCR CSKCCEDLEE
651 AQEGQDVPVK APETFDNITI SREAQGEVPA
        SDSKTECTAL
```

Antibodies disclosed herein specifically bind to an epitope on an extracellular domain (ECD) of the human NaPi2b.

Those skilled in the art will recognize that it is possible to determine, without undue experimentation, if a monoclonal antibody has the same specificity as a monoclonal antibody disclosed herein (e.g., XMT-1535) by ascertaining whether the former prevents the latter from binding to a natural binding partner or other molecule known to be associated with NaPi2b. If the monoclonal antibody being tested competes with the monoclonal antibody disclosed herein, as shown by a decrease in binding by the monoclonal antibody disclosed herein, then the two monoclonal antibodies bind to the same, or a closely related, epitope.

An alternative method for determining whether a monoclonal antibody has the specificity of monoclonal antibody disclosed herein is to pre-incubate the monoclonal antibody disclosed herein with soluble NaPi2b (with which it is normally reactive), and then add the monoclonal antibody being tested to determine if the monoclonal antibody being tested is inhibited in its ability to bind NaPi2b. If the monoclonal antibody being tested is inhibited then, in all likelihood, it has the same, or functionally equivalent, epitopic specificity as the monoclonal antibody disclosed herein.

Screening of monoclonal antibodies disclosed herein, can also be carried out, e.g., by measuring NaPi2b-mediated activity, and determining whether the test monoclonal antibody is able to modulate, block, inhibit, reduce, antagonize, neutralize or otherwise interfere with NaPi2b activity.

The antibodies disclosed herein contain a heavy chain variable region having an amino acid sequence at least 90%, 91%, 92%, 93%, 94%, 95%, 96%, 97% 98%, 99% or more identical to a sequence consisting of SEQ ID NO: 3 and a light chain variable region having an amino acid sequence at least 90%, 91%, 92%, 93%, 94%, 95%, 96%, 97% 98%, 99% or more identical to a sequence consisting of SEQ ID NO: 4.

In some embodiments, the antibodies disclosed herein contain a heavy chain amino acid sequence at least 90%, 91%, 92%, 93%, 94%, 95%, 96%, 97% 98%, 99% or more identical to the amino acid sequence of SEQ ID NO: 1 and a light chain amino acid sequence at least 90%, 91%, 92%, 93%, 94%, 95%, 96%, 97% 98%, 99% or more identical to the amino acid sequence of SEQ ID NO: 2.

The antibodies disclosed herein contain a heavy chain variable region having an amino acid sequence at least 85%, 86%, 87% 88%, 89%, 90%, 91%, 92%, 93%, 94%, 95%, 96%, 97% 98%, 99% or more identical to a sequence consisting of SEQ ID NO: 3 and a light chain variable region having an amino acid sequence at least 85%, 86%, 87% 88%, 89%, 90%, 91%, 92%, 93%, 94%, 95%, 96%, 97% 98%, 99% or more identical to a sequence consisting of SEQ ID NO: 4.

In some embodiments, the antibodies disclosed herein contain a heavy chain amino acid sequence at least 85%, 86%, 87% 88%, 89%, 90%, 91%, 92%, 93%, 94%, 95%, 96%, 97% 98%, 99% or more identical to the amino acid sequence of SEQ ID NO: 1 and a light chain amino acid sequence at least 85%, 86%, 87% 88%, 89%, 90%, 91%, 92%, 93%, 94%, 95%, 96%, 97% 98%, 99% or more identical to the amino acid sequence of SEQ ID NO: 2.

In some embodiments, the antibodies disclosed herein contain the heavy chain variable region amino acid sequence of SEQ ID NO: 3 and the light chain variable region amino acid sequence of SEQ ID NO: 4.

In some embodiments, the antibodies disclosed herein contain the heavy chain amino acid sequence of SEQ ID NO: 1 and the light chain amino acid sequence of SEQ ID NO: 2.

In some embodiments, the antibodies disclosed herein contain the CDRH1 amino acid sequence of SEQ ID NO: 5, the CDRH2 amino acid sequence of SEQ ID NO: 6, the CDRH3 amino acid sequence of SEQ ID NO: 7, the CDRL1 amino acid sequence of SEQ ID NO: 8, the CDRL2 amino acid sequence of SEQ ID NO: 9, and the CDRL3 amino acid sequence of SEQ ID NO: 10.

In some embodiments, the antibodies disclosed herein that contains the amino acid sequence at least 90%, 91%, 92%, 93%, 94%, 95%, 96%, 97% 98%, 99% or more identical to the amino acid sequence GYTFTGYNIH (SEQ ID NO: 5); a CDRH2 that contains the amino acid sequence at least 90%, 91%, 92%, 93%, 94%, 95%, 96%, 97% 98%, 99% or more identical to the amino acid sequence AIYPGNGDTSYKQKFRG (SEQ ID NO: 6); a CDRH3 that contains the amino acid sequence at least 90%, 91%, 92%, 93%, 94%, 95%, 96%, 97% 98%, 99% or more identical to the amino acid sequence GETARATFAY (SEQ ID NO: 7); a CDRL1 that contains the amino acid sequence at least 90%, 91%, 92%, 93%, 94%, 95%, 96%, 97% 98%, 99% or more identical to the amino acid sequence SASQDIGNFLN (SEQ ID NO: 8); a CDRL2 that contains the amino acid sequence at least 90%, 91%, 92%, 93%, 94%, 95%, 96%, 97% 98%, 99% or more identical to the amino acid sequence YTSSLYS (SEQ ID NO: 9); and a CDRL3 that contains the amino acid sequence at least 90%, 91%, 92%, 93%, 94%, 95%, 96%, 97% 98%, 99% or more identical to the amino acid sequence QQYSKLPLT (SEQ ID NO: 10).

In some embodiments, the antibodies disclosed herein that contains the amino acid sequence at least 85%, 86%, 87% 88%, 89%, 90%, 91%, 92%, 93%, 94%, 95%, 96%, 97% 98%, 99% or more identical to the amino acid sequence GYTFTGYNIH (SEQ ID NO: 5); a CDRH2 that contains the amino acid sequence at least 85%, 86%, 87% 88%, 89%, 90%, 91%, 92%, 93%, 94%, 95%, 96%, 97% 98%, 99% or more identical to the amino acid sequence AIYPGNGDTSYKQKFRG (SEQ ID NO: 6); a CDRH3 that contains the amino acid sequence at least 85%, 86%, 87% 88%, 89%, 90%, 91%, 92%, 93%, 94%, 95%, 96%, 97% 98%, 99% or more identical to the amino acid sequence GETARATFAY (SEQ ID NO: 7); a CDRL1 that contains the amino acid sequence at 85%, 86%, 87% 88%, 89%, 90%, 91%, 92%, 93%, 94%, 95%, 96%, 97% 98%, 99% or more identical to the amino acid sequence SASQDIGNFLN (SEQ ID NO: 8); a CDRL2 that contains the amino acid sequence at least 85%, 86%, 87% 88%, 89%, 90%, 91%, 92%, 93%, 94%, 95%, 96%, 97% 98%, 99% or more identical to the amino acid sequence YTSSLYS (SEQ ID NO: 9); and a CDRL3 that contains the amino acid sequence at least 85%, 86%, 87% 88%, 89%, 90%, 91%, 92%, 93%, 94%, 95%, 96%, 97% 98%, 99% or more identical to the amino acid sequence QQYSKLPLT (SEQ ID NO: 10).

In certain embodiments, the antibodies disclosed herein include one or more conservative amino acid substitutions in a variable domain sequence such as 1, 2, 3, 4, 5, 6, 7, 8, 9, 10, 11, 12, 13, 14, 15, or more conservative substitutions in a variable domain sequence. In some embodiments, these conservative amino acid substitutions are in a CDR region, e.g., 1, 2, 3, 4, 5, 6, 7, 8, 9, 10, 11, 12, 13, 14, 15, or more conservative substitutions are made cumulatively across all CDRs and in some particular embodiments, up to 1, 2, 3, or 4 conservative amino acid substitutions may be present in each CDR sequence, e.g., SEQ ID NOs: 5-10.

Those skilled in the art will recognize that it is possible to determine, without undue experimentation, if a monoclonal antibody has the same specificity as a monoclonal antibody XMT-1535, by ascertaining whether the former prevents the latter from binding to a natural binding partner or other molecule known to be associated with NaPi2b. If the monoclonal antibody being tested competes with the monoclonal antibody disclosed herein, as shown by a decrease in binding by the monoclonal antibody disclosed herein, then the two monoclonal antibodies bind to the same, or a closely related, epitope.

An alternative method for determining whether a monoclonal antibody has the specificity of monoclonal antibody disclosed herein is to pre-incubate the monoclonal antibody disclosed herein with soluble NaPi2b (with which it is normally reactive), and then add the monoclonal antibody being tested to determine if the monoclonal antibody being tested is inhibited in its ability to bind NaPi2b. If the monoclonal antibody being tested is inhibited then, in all likelihood, it has the same, or functionally equivalent, epitopic specificity as the monoclonal antibody disclosed herein.

Screening of monoclonal antibodies disclosed herein, can be also carried out, e.g., by measuring NaPi2b-mediated activity, and determining whether the test monoclonal antibody is able to modulate, block, inhibit, reduce, antagonize, neutralize or otherwise interfere with NaPi2b activity.

The NaPi2b antibodies suitable for use in the methods disclosed herein can be generated and purified by well-known techniques e.g., WO 2009/097128, WO 2017/160754, and U.S. Ser. No. 16/136,706, each of which is incorporated herein in its entirety by reference.

### NaPi2b-Targeted Polymer Antibody Drug Conjugates

The invention pertains to therapies involving immunoconjugates comprising an antibody conjugated to a cytotoxic agent such as a toxin (e.g., an enzymatically active toxin of bacterial, fungal, plant, or animal origin, or fragments thereof), via a polymer scaffold.

The conjugate described herein includes a NaPi2b antibody connected to one or more AF-HPA-carrying polymeric scaffolds independently comprising poly(1-hydroxymethylethylene hydroxymethyl-formal) (PHF) having a molecular weight ranging from about 5 kDa to about 10 kDa. The AF-HPA-carrying polymeric scaffold is conjugated to the NaPi2b-targeted antibody via the NaPi2b cysteine residues.

Specifically, the NaPi2b-targeted polymer antibody-drug conjugate is XMT-1536 and has the Formula (A):

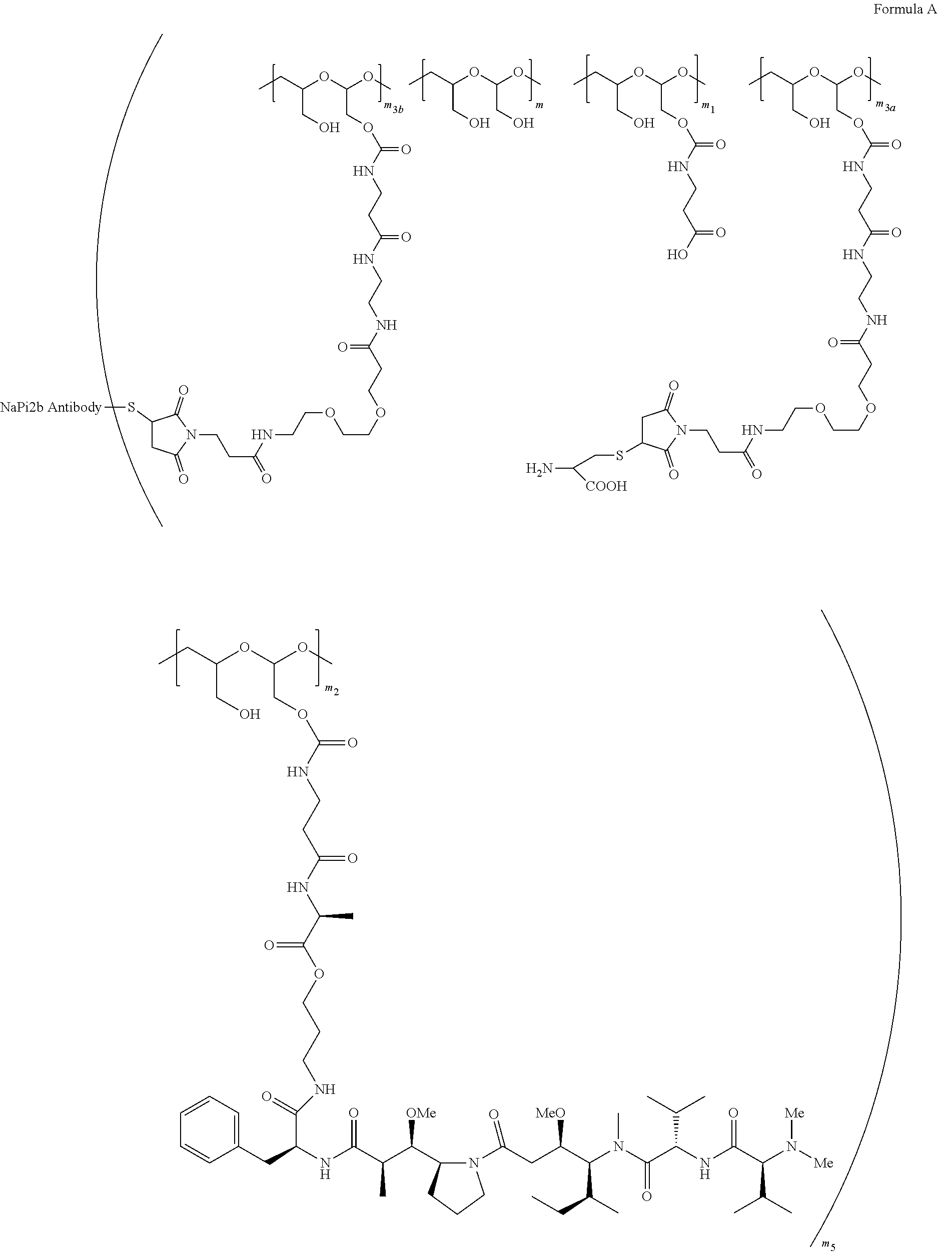

wherein:
the polymer comprises poly(1-hydroxymethylethylene hydroxymethyl-formal) (PHF) having a molecular weight ranging from about 5 kDa to about 10 kDa;
m is an integer from 20 to 75,
m₁ is an integer from about 5 to about 35,
m₂ is an integer from about 3 to about 10,
m₃ₐ is an integer from 0 to about 4,
m_{3b} is an integer from 1 to about 5,
the sum of m, m₁, m₂, m₃ₐ, and m_{3b} ranges from about 40 to about 75,
m₅ is an integer from about 2 to about 6, and
   NaPi2b is the fully human or humanized NaPi2b antibody XMT1535 described herein.

In some embodiments, m is an integer from about 30 to about 75.

In some embodiments, m is an integer from about 30 to about 40.

In some embodiments, m₁ is an integer from about 10 to about 20.

In some embodiments, m₁ is an integer from about 10 to about 12.

In some embodiments, m₂ is an integer from about 3 to about 5.

In some embodiments, m₃ₐ is an integer from 0 to about 1.

In some embodiments, m_{3b} is an integer from 2 to about 4

In some embodiments, m₅ is an integer from about 2 to about 5.

In some embodiments, m₅ is an integer from about 2 to about 4.

In some embodiments, m₅ is an integer from about 3 to about 4.

In some embodiments the NaPi2b-targeted polymer antibody-drug conjugate comprises 10 to 15 molecules of AF-HPA.

In some embodiments, the PHF has a molecular weight ranging from about 6 kDa to about 8 kDa.

In some embodiments, the PHF has a molecular weight ranging from about 6 kDa to about 7 kDa.

In certain embodiments, the NaPi2b-targeted polymer antibody-drug conjugate Formula (A) is of Formula (B), wherein the polymer is PHF that has a molecular weight ranging from about 5 kDa to about 10 kDa:

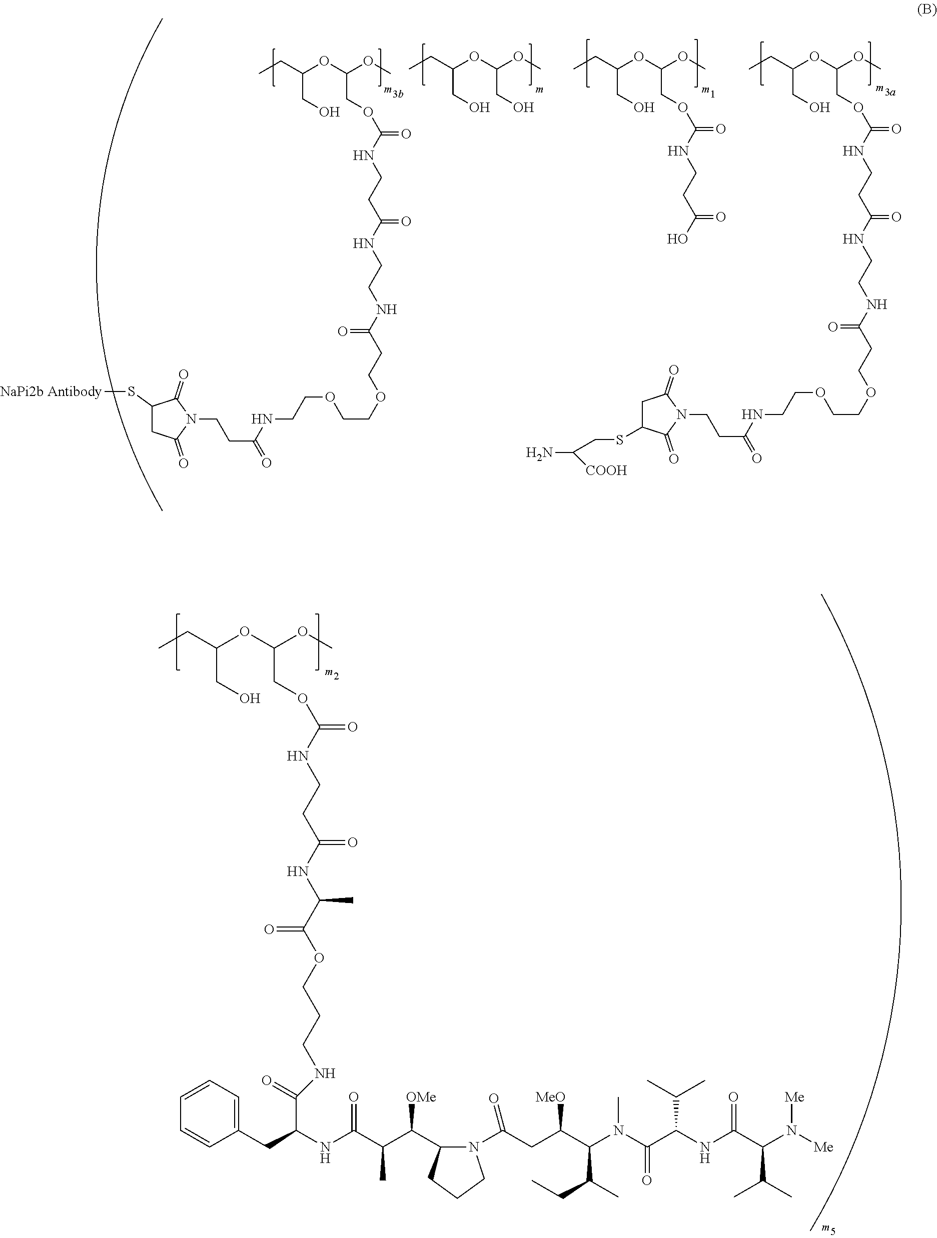

wherein:
m is an integer from 30 to about 35,
m₁ is an integer from 8 to about 10,
m₂ is an integer from 2 to about 5,
m₃ₐ is an integer from 0 to about 1,
m_{3b} is an integer from 1 to about 2,
the sum of m₃ₐ and m_{3b} ranges from 1 and about 4, and
m₅ is an integer from about 2 to about 6.

The NaPi2b-targeted polymer antibody-drug conjugates, (i.e., XMT-1536) suitable for use in the methods disclosed herein can be generated and purified by well-known techniques e.g., WO 2009/097128, WO 2017/160754, PCT/US18/38988 and U.S. Ser. No. 16/136,706, each of which is incorporated herein in its entirety by reference.

### Dosage and Administration

Patients with recurrent, platinum-sensitive, ovarian are administered a NaPi2b-targeted polymer antibody-drug conjugate (XMT-1536) in an amount sufficient to exert a therapeutically useful effect. The ovarian cancer includes, but is not limited to, fallopian tube cancer and primary peritoneal cancer. In some aspects, the ovarian cancer is high-grade serous ovarian cancer (HGSOC).

In some embodiments, the subject has platinum-sensitive HGSOC. In some embodiments the subject has fallopian tube cancer. In other embodiments, the subject has primary peritoneal cancer. The platinum-sensitive HGSOC can be recurrent. In some embodiments the subject has recurrent high grade serous ovarian cancer, that includes fallopian tube cancer and primary peritoneal cancer.

In some embodiments, the subject must have received 4 to 8 cycles of platinum-based chemotherapy in 2^{nd} to 4^{th} line setting. In some embodiments, the patient has received 4, 5, 6, 7, or 8 cycles of platinum-based chemotherapy. In some embodiments, the patient has received 4, 5, 6, 7, or 8 cycles of platinum-based combination chemotherapy. These platinum-based combination chemotherapy regimens administered immediately prior to the administration of XMT-1536, include, but are not limited to, carboplatin or cisplatin with or without paclitaxel, docetaxel, pegylated liposomal doxorubicin or gemcitabine.

In some embodiments, the subject must have received 4 to 8 cycles of platinum-based chemotherapy in 2^{nd} to 4^{th} line setting has no evidence of disease (NED)/complete response (CR)/partial response (PR)/or stable disease (SD) as best response.

In some embodiments, the subject has a recurrent, platinum-sensitive HGSOC (including fallopian tube and primary peritoneal cancer) and has stable disease as best response to their most recent platinum-based regimen prior to the administration of XMT-1536.

In some embodiments, the subject having platinum-sensitive recurrent disease is administered XMT-1536 between 3 and 12 weeks after completion of the final dose of platinum in the most recent platinum-based regimen.

In some embodiments, the subject having known deleterious somatic or germline BRCA gene mutation must have received prior PARP inhibitor therapy prior to the administration of XMT-1536. In some embodiment, the PARP inhibitor, includes, but is not limited to, niraparib, rucaparib and olaparib.

In some embodiments the subject who has received only one prior line of platinum-based therapy or who has received bevacizumab in combination with their most recent platinum-based regimen is excluded from the administration or treatment with XMT-1536.

In some aspects, the use of a NaPi2b-targeted antibody polymer-drug conjugate prevents progression of the ovarian cancer. In some aspects, the use of a NaPi2b-targeted antibody polymer-drug conjugate delays the progression of the ovarian cancer relative to treatment with a placebo. In some aspects, the use of a NaPi2b-targeted antibody polymer-drug conjugate delays the progression of the ovarian cancer relative to treatment with a different therapeutic agent.

In some embodiments, the NaPi2b-targeted antibody polymer-drug conjugate is administered as a maintenance therapy following an initial treatment with a platinum-containing agent such as carboplatin. In some aspects, the use of a NaPi2b-targeted antibody polymer-drug conjugate as a maintenance therapy prevents progression of the ovarian cancer. In some aspects, the use of a NaPi2b-targeted antibody polymer-drug conjugate as a maintenance therapy delays the progression of the ovarian cancer relative to treatment with a placebo. In some aspects, the use of a NaPi2b-targeted antibody polymer-drug conjugate as a maintenance therapy delays the progression of the ovarian cancer relative to treatment with a different therapeutic agent.

In some embodiments, the subject experiences improved progression-free survival following treatment with the NaPi2b-targeted antibody polymer-drug conjugate relative to treatment with placebo.

In some embodiments, administration of XMT-1536 is via infusion. Methods of infusion can comprise any method of infusing therapeutic agents to a subject known in the art. In some embodiments the infusion is an intravenous (IV) infusion.

In some embodiments, infusions of XMT-1536 occur over a duration of at least 1 minute, at least 5 minutes, at least 10 minutes, at least 15 minutes, at least 20 minutes, at least 25 minutes, at least 30 minutes, at least 35 minutes, at least 45 minutes, at least 50 minutes, at least 55 minutes, at least 60 minutes, at least 65 minutes, at least 70 minutes, at least 75 minutes, at least 80 minutes, at least 85 minutes, at least 90 minutes, at least 95 minutes, at least 100 minutes, at least 105 minutes, at least 110 minutes, at least 115 minutes, at least 120 minutes, or any number of minutes therebetween. In some embodiments, the duration of infusion can be varied from the first infusion to the second and subsequent infusions.

In some embodiments, the subject having platinum-sensitive HGSOC is administered by infusion, XMT-1536 at a dosage amount that is between about 20 mg/m² to 43 mg/m². For example, the dosage of XMT-1536 is 20 mg/m². For example, the dosage of XMT-1536 is 25 mg/m². Alternatively, the dosage of XMT-1536 is 30 mg/m². In some embodiments, the dosage of XMT-1536 is 36 mg/m². In other embodiments, the dosage of XMT-1536 is 43 mg/m². In some embodiments, the dosage of XMT-1536 is 20 mg/m² is capped at BSA 2.2 m². In some embodiments, the dosage of XMT-1536 is 25 mg/m² is capped at BSA 2.2 m². In some embodiments, the dosage of XMT-1536 is 30 mg/m² is capped at BSA 2.2 m². In some embodiments, the dosage of XMT-1536 is 36 mg/m² is capped at BSA 2.2 m². In some embodiments, the dosage of XMT-1536 is 43 mg/m² is a capped at BSA 1.8 m². In some embodiments, the dosage of XMT-1536 is 36 mg/m² up to a maximum of approximately 80 mg. In some embodiments, the dosage of XMT-1536 is 30 mg/m² up to a maximum of approximately 80 mg. In some embodiments, the dosage of XMT-1536 is 25 mg/m² up to a maximum of approximately 80 mg. In some embodiments, the dosage of XMT-1536 is about 80 mg. In these embodiments the dosage amounts are administered intravenously once every four weeks i.e. 28-day cycle.

In some embodiments the subject is administered XMT-1536 over 90 min for the first infusion, then over 30 minutes for the subsequent infusions for up to 18 cycles once every 4 weeks.

In some embodiments the subject is administered XMT-1536 at a dosage of 20 mg/m² over 90 min for the first infusion, then over 30 minutes for the subsequent infusions for up to 18 cycles once every 4 weeks.

In some embodiments the subject is administered XMT-1536 at a dosage of 30 mg/m² over 90 min for the first infusion, then over 30 minutes for the subsequent infusions for up to 18 cycles once every 4 weeks.

In some embodiments the subject is administered XMT-1536 at a dosage of 20 mg/m² over 90 min for the first infusion, then over 30 minutes for the subsequent infusions for up to 18 cycles once every 4 weeks. In some embodiments, the dosage of XMT-1536 is 20 mg/m² capped at BSA 2.2 m².

In some embodiments the subject is administered XMT-1536 at a dosage of 25 mg/m² over 90 min for the first infusion, then over 30 minutes for the subsequent infusions for up to 18 cycles once every 4 weeks. In some embodiments, the dosage of XMT-1536 is 25 mg/m² capped at BSA 2.2 m².

In some embodiments the subject is administered XMT-1536 at a dosage of 30 mg/m² over 90 min for the first infusion, then over 30 minutes for the subsequent infusions for up to 18 cycles once every 4 weeks. In some embodiments, the dosage of XMT-1536 is 30 mg/m² capped at BSA 2.2 m².

In some embodiments the subject is administered XMT-1536 at a dosage of 36 mg/m² over 90 min for the first infusion, then over 30 minutes for the subsequent infusions for up to 18 cycles once every 4 weeks. In some embodiments, the dosage of XMT-1536 is 36 mg/m² is capped at BSA 2.2 m².

In some embodiments the subject is administered XMT-1536 at a dosage of 43 mg/m² over 90 min for the first infusion, then over 30 minutes for the subsequent infusions for up to 18 cycles once every 4 weeks. In some embodiments, the dosage of XMT-1536 is 43 mg/m² is a capped at BSA 1.8 m².

In some embodiments the subject is administered XMT-1536 at a dose of about 80 mg over 90 min for the first infusion, then over 30 minutes for the subsequent infusions for up to 18 cycles once every 4 weeks.

In some embodiments, the subject is administered XMT-1536 for 18 months or until disease progression, death, unacceptable toxicity or voluntary withdrawal—whichever comes first.

In some embodiments, the XMT-1536 infusions at a dosage amount that is between about 20 mg/m² to 43 mg/m² is administered as an infusion every one week, every two weeks, every three weeks, every four weeks, every five weeks, every six weeks, every seven weeks, or every eight weeks.

### Dose Reductions and Delays

In some cases, toxicity or adverse reactions may occur in response to the initial dose of XMT-1536 at the doses descried herein. Subsequent doses may be reduced or time between doses may be delayed or extended. In some embodiments, toxicity or adverse reactions that may result in a dose reduction or delay include, but are not limited to, hepatotoxicity, interstitial lung disease (ILD), pneumonitis, hematologic toxicity, proteinuria, fatigue, nausea, other clinically significant non-laboratory toxicities, other clinically significant adverse events.

For delays in dosing, the delay can occur for up to 8 weeks.

An exemplary dose reduction protocol wherein the dose of XMT-1536 is reduced is presented below.

| | upifitamab rilsodotin |
|---|---|
| Level | (mg/m², BSA capped at 2.2 m²) |
| Starting dose | 30 |
| 1^{st} incremental reduction | 25 |
| 2^{nd} incremental reduction | 20 |
| 3^{rd} incremental reduction | Discontinue |

In some embodiments, the dose is reduced following the first infusion. In some embodiments, the dose is reduced following subsequent infusions.

### Measurement of NaPi2b Expression

In various embodiments the invention provides a method for identifying a cancer patient amenable to NaPi2b-targeted therapy or monitoring the treatment regimen by measuring the status of NaPi2b expression in a tumor sample obtained from the patient.

In some embodiments, the NaPi2b diagnostic tests can be used to identification subjects for treatment with the NaPi2b-targeted polymer drug conjugate.

The sample is derived from the subject having a cancer. The sample of cancer cells is dissected from tissue removed or obtained from the subject. In some embodiments, the sample is a fresh, frozen or an archival biopsy sample.

In some embodiments, the test cell population is derived from fresh, unfrozen tissue from a biopsy sample. In other embodiments, the test cell population is derived from a primary or metastatic site. In some embodiments, the test cell population is derived from a fresh or frozen tissue from a biopsy or surgical sample or ascitic fluid or pleural fluid. In some embodiments, the test cell population is derived from a fixed tissue (e.g., formalin fixation or formalin-fixed paraffin-embedded (FFPE)) from a biopsy or surgical sample or cell block derived from a fluid specimen. The tissue sample may be frozen or fresh.

The requisite level of NaPi2b expression may be that which is identified by the any methods known in the art and more specifically by the methods described herein. For example, the level of NaPi2b expression can be measured by conducting a known immunological assay, such as an enzyme immunoassay, radioimmunoassay, competitive immunoassay, double antibody sandwich assay, fluoroimmuno assay, ELISA, Western blotting technique, agglutination assay, cytofluorometry (e.g. flow cytometry), Fluorescence in situ hybridization (FISH), colorimetric or immunohistochemical staining assay (IHC) for protein expression using an antibody that specifically recognizes NaPi2b. Cell-based assays, such as, for example, flow cytometry (FC), immuno-histochemistry (IHC), RNA expression analysis or immunofluorescence (IF) are particularly desirable in determining NaPi2b expression status, since such assay formats are clinically-suitable.

Flow cytometry (FC) may be employed to determine cell surface expression of NaPi2b in a tumor sample before, during, and after treatment with a drug. For example, tumor cells may be analyzed by flow cytometry for NaPi2b expression, as well as for markers identifying cancer cell types, etc., if so desired. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 2% paraformaldehyde for 10 minutes at 37° C. followed by permeabilization in 90% methanol for 30 minutes on ice. Cells may then be stained with NaPi2b-specific antibody, washed and labeled with a fluorescent-labeled secondary antibody. The cells would then be analyzed on a flow cytometer (e.g. a Beckman Coulter FC500) according to the specific protocols of the instrument used. Such an analysis would identify the level of expressed NaPi2b in the tumor.

Immunohistochemical (IHC) staining may be also employed to determine the expression of NaPi2b in a tumor sample before, during, and after treatment with a drug. IHC may be carried out according to well-known techniques. See, e.g., ANTIBODIES; A LABORATORY MANUAL, Chapter 10, Harlow & Lane Eds., Cold Spring Harbor Laboratory (1988). Briefly, and by way of example, paraffin-embedded tissue (e.g. tumor tissue from a biopsy) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary polypeptide antibody and secondary antibody; and finally detecting using ABC avidin/biotin method according to manufacturer's instructions.

Immunofluorescence (IF) assays may be also employed to determine the expression of NaPi2b tumor sample before, during, and after treatment with a drug. IF may be carried out according to well-known techniques. See, e.g., J. M. Polak and S. Van Noorden (1997) INTRODUCTION TO IMMUNOCYTOCHEMISTRY, 2nd Ed.; ROYAL MICROSCOPY SOCIETY MICROSCOPY HANDBOOK 37, BioScientific/Springer-Verlag. Briefly, and by way of example, patient samples may be fixed in paraformaldehyde followed by methanol, blocked with a blocking solution such as horse serum, incubated with the primary antibody against polypeptide followed by a secondary antibody labeled with a fluorescent dye such as Alexa 488 and analyzed with an epifluorescent microscope.

Antibodies employed in the above-described assays may be advantageously conjugated to fluorescent dyes (e.g. Alexa488, PE), or other labels, such as quantum dots, for use in multi-parametric analyses along with other signal transduction (phospho-AKT, phospho-Erk 1/2) and/or cell marker (cytokeratin) antibodies.

In one embodiment the expression of NaPi2b in a sample from a tumor is determined immunohistochemically. In another embodiment, the expression of NaPi2b in a sample from a tumor is determined immunohistochemically (IHC) using the method described in U.S. Ser. No. 16/136,706, which is incorporated herein in its entirety by reference. In another embodiment, the expression of NaPi2b in a sample from a tumor is determined using a system such as, for example, a Leica BOND-III Fully Automated Stainer (BOND-III) system. Briefly the assay system is comprised of the following (1) a detection antibody also known as the IHC antibody (2) the IHC Platform i.e. the BOND-III instrument, with an established protocol for pre-treatment, epitope retrieval and staining, as well as pre-specified control material and (3) a defined scoring method, as described in a scoring and interpretation guide.

Alternatively, the assay may include preparing RNA from the sample, optionally for use in PCR (polymerase chain reaction) or other analytical methodology. The PCR methodology is optionally, for example, RT-PCR (reverse transcription-PCR) or quantitative PCR, such as, for example, real-time RT-PCR, RNA seq and the like. Alternatively, the assaying may be conducted by use of an array, such as a microarray as known in the relevant field, such as, for example, nanostring technologies.

Patients are identified as being responsive to treatment, wherein the treatment is monitored or cancer is detected by detecting and/or measuring the expression level of NaPi2b in the tumor cells in a sample.

The detection/measurement of the expression level of NaPi2b is determined by calculating a NaPi2b score. The NaPi2b score is quantitative or semi quantitative. For example, detection is scored pathologically to arrive at a pathology score. It is contemplated that any scoring methods known in the art may be used in the methods of the invention. In particular, any histological scoring methods known in the art.

The methods for assessing the measurement results obtained by immunohistochemical staining assays include, for example, the H-score method, TPS (tumor proportion score) or PS2+(percent score) score. The H-score (Am J Clin Pathol. 1988; 90 (3): 233-9), TPS score and PS2+scores are determined by the following calculation formula. H-Score=((% at 0)×0)+((% at 1+)×1)+((% at 2+)×2)+((% at 3+)×3), TPS-score=(% at 1+)+(O % at 2+)+(% at 3+); and PS2+score=(% at 2+)+(% at 3+); where staining intensity 0 is unstained; staining intensity 1 is weak staining; staining intensity 2 is moderate staining; and staining intensity 3 is strong staining. In some embodiments the subject having a TPS of ≥75 will be considered NaPi2b positive (high) in this assay and a TPS of <75 will be considered NaPi2b negative (low) when TPS is scored as tumor cell membrane reactivity.

In assessment by the scoring method, only cancer cell portions are used. For negative or positive controls for staining intensity, formalin-fixed paraffin-embedded cell lines or xenografts (lines whose protein expression levels are known in advance) may be employed. When there are no control specimens, a plurality of specimens are assessed simultaneously to confirm the overall distribution of staining intensity of the specimens, and then staining intensity may be set.

In addition to the scoring methods mentioned above, other scoring methods known in the art, such as, for example, the Allred method (Harvey, et al. Journal of Clinical Oncology 17, No. 5 (May 1999) 1474-1474), can also be used. Cut-off points are required to be set in each method. Allred score=score of percentage of positive cells+staining intensity score.

The disclosure also provides kits and/or methods for identifying or otherwise refining, e.g., stratifying, a patient population suitable for therapeutic administration of a NaPi2b-targeted antibody-drug conjugates disclosed herein by identifying the NaPi2b score of the subject prior to treatment with a NaPi2b-targeted antibody-drug conjugate disclosed herein. In some embodiments, the test cell population is derived from fresh, unfrozen tissue from a biopsy sample. In some embodiments, the test cell population is derived from a primary or metastatic site. In some embodiments, the test cell population is derived from a frozen tissue from a biopsy or surgical sample or ascetic fluid or pleural fluid. In some embodiments, the test cell population is derived from a fixed tissue (e.g., formalin fixation) from a biopsy or surgical sample. The IHC test measures the amount of NaPi2b receptor protein on the surface of cells in a cancer tissue sample

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the terms “NaPi2b” (also known as sodium-dependent phosphate transport protein 2B, SLC34A2, NaPiIIb, Npt2, Na(+)-dependent phosphate cotransporter 2B; sodium/phosphate cotransporter 2B; Na(+)/Pi cotransporter 2B; NaPi3b; solute carrier family 34 member 2), when used herein, refers to human NaPi2b (e.g., GenBank Accession No. O95436.3) and includes any variants, isoforms and species homologs of NaPi2b which are naturally expressed by cells, including tumor cells, or are expressed on cells transfected with the NaPi2b gene. These terms are synonymous and may be used interchangeably.

As used herein, the term “NaPi2b antibody” or “anti-NaPi2b antibody” is an antibody which binds specifically to the extracellular region of SLC34A2.

When used herein in the context of two or more antibodies, the term “competes with” or “cross-competes with” indicates that the two or more antibodies compete for binding to NaPi2b, e.g., compete for NaPi2b binding in any art-recognized assay. An antibody “blocks” or “cross-blocks” one or more other antibodies from binding to NaPi2b if the antibody competes with the one or more other antibodies 25% or more, with 25%-74% representing “partial block” and 75%-100% representing “full block”, as determined using any art-recognized assay. For some pairs of antibodies, competition or blocking in any art-recognized assay is only observed when one antibody is coated on the plate and the other is used to compete, and not vice versa. Unless otherwise defined or negated by context, the terms “competes with”, “cross-competes with”, “blocks” or “cross-blocks” when used herein is also intended to cover such pairs of antibodies

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” “or directed against” is meant that the antibody reacts with one or more antigenic determinants of the desired antigen and does not react with other polypeptides or binds at much lower affinity (K_{d}>10⁻⁶). Antibodies include, but are not limited to, polyclonal, monoclonal and chimeric antibodies.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “monoclonal antibody” (mAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. mAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin or fragment thereof, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the dissociation constant is ≤1 μM; e.g., ≤100 nM, preferably ≤10 nM and more preferably ≤1 nM.

The term “polypeptide” is used herein as a generic term to refer to native protein, fragments, or analogs of a polypeptide sequence. Hence, native protein fragments, and analogs are species of the polypeptide genus. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Green, Eds., Sinauer Associates, Sunderland7 Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present disclosure. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end the left-hand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and which are 5′ to the 5′ end of the RNA transcript are referred to as “upstream sequences”, sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present disclosure, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the disclosure.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986), Veber and Freidinger TINS p. 392 (1985); and Evans et al. J. Med. Chem. 30:1229 (1987). Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992)); for example, by adding internal cysteine residues capable of forming intramolecular disulfide bridges which cyclize the peptide.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ³¹¹I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The use of the articles “a”, “an”, and “the” in both the following description and claims are to be construed to cover both the singular and the plural, unless otherwise indicated herein or clearly contradicted by context. The terms “comprising”, “having”, “being of” as in “being of a chemical formula”, “including”, and “containing” are to be construed as open terms (i.e., meaning “including but not limited to”) unless otherwise noted. For example, a polymeric scaffold of a certain formula includes all the monomer units shown in the formula and may also include additional monomer units not shown in the formula. Additionally, whenever “comprising” or another open-ended term is used in an embodiment, it is to be understood that the same embodiment can be more narrowly claimed using the intermediate term “consisting essentially of” or the closed term “consisting of.”

The term “about”, “approximately”, or “approximate”, when used in connection with a numerical value, means that a collection or range of values is included. For example, “about X” includes a range of values that are ±20%, ±10%, ±5%, ±2%, ±1%, ±0.5%, ±0.2%, or ±0.1% of X, where X is a numerical value. In one embodiment, the term “about” refers to a range of values which are 5% more or less than the specified value. In another embodiment, the term “about” refers to a range of values which are 2% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the expressions “x being an integer between 1 and 6” and “x being an integer of 1 to 6” both mean “x being 1, 2, 3, 4, 5, or 6”, i.e., the terms “between X and Y” and “range from X to Y, are inclusive of X and Y and the integers there between.

All methods described herein can be performed in any suitable order unless otherwise indicated herein or otherwise clearly contradicted by context. The use of any and all examples, or exemplary language (e.g., “such as”) provided herein, is intended merely to better illustrate the invention and is not to be construed as a limitation on the scope of the claims unless explicitly otherwise claimed. No language in the specification is to be construed as indicating that any non-claimed element is essential to what is claimed.

“Polymeric Carrier or scaffold”: The term polymeric carrier or scaffold, as used herein, refers to a polymer or a modified polymer, which is suitable for covalently attaching to or can be covalently attached to one or more drug molecules with a designated linker and/or one or more PBRMs with a designated linker.

“Physiological conditions”: The phrase “physiological conditions”, as used herein, relates to the range of chemical (e.g., pH, ionic strength) and biochemical (e.g., enzyme concentrations) conditions likely to be encountered in the extracellular fluids of living tissues. For most normal tissues, the physiological pH ranges from about 7.0 to 7.4. Circulating blood plasma and normal interstitial liquid represent typical examples of normal physiological conditions.

“Drug”: As used herein, the term “drug” refers to a compound which is biologically active and provides a desired physiological effect following administration to a subject in need thereof (e.g., an active pharmaceutical ingredient).

“Cytotoxic”: As used herein the term “cytotoxic” means toxic to cells or a selected cell population (e.g., cancer cells). The toxic effect may result in cell death and/or lysis. In certain instances, the toxic effect may be a sublethal destructive effect on the cell, e.g., slowing or arresting cell growth. In order to achieve a cytotoxic effect, the drug or prodrug may be selected from a group consisting of a DNA damaging agent, a microtubule disrupting agent, or a cytotoxic protein or polypeptide, amongst others.

“PHF” refers to poly(1-hydroxymethylethylene hydroxymethyl-formal).

As used herein, the terms “polymer unit”, “monomeric unit”, “monomer”, “monomer unit”, “unit” all refer to a repeatable structural unit in a polymer.

As used herein, “molecular weight” or “MW” of a polymer or polymeric carrier/scaffold or polymer conjugates refers to the weight average molecular weight of the unmodified polymer unless otherwise specified.

As used herein, “dosing regimen” or “dosage regimen” refers to the amount of agent, for example, the composition containing an NaPi2b-targeted polymer antibody-drug conjugate, administered, and the frequency of administration. The dosing regimen is a function of the disease or condition to be treated, and thus can vary.

As used herein, “frequency” of administration refers to the time between successive administrations of treatment. For example, frequency can be days, weeks or months. For example, frequency can be more than once weekly, for example, twice a week, three times a week, four times a week, five times a week, six times a week or daily. Frequency also can be one, two, three or four weeks. The particular frequency is a function of the particular disease or condition treated. Generally, frequency is more than once weekly, and generally is twice weekly.

As used herein, a “cycle of administration” refers to the repeated schedule of the dosing regimen of administration of the enzyme and/or a second agent that is repeated over successive administrations. For example, an exemplary cycle of administration is a 28-day cycle with administration twice weekly for three weeks, followed by one-week of discontinued dosing. A preferred cycle of administration is a 21-day cycle with administration once every 21 days (i.e., 3 weeks) or a 28 day cycle with administration once every 28 days (i.e., 4 weeks)

As used herein, when referencing dosage based on mg/kg of the subject, an average human subject is considered to have a mass of about 70 kg-75 kg, such as 70 kg and a body surface area (BSA) of 1.73 m².

As used herein, amelioration of the symptoms of a particular disease or disorder by a treatment, such as by administration of a pharmaceutical composition or other therapeutic, refers to any lessening, whether permanent or temporary, lasting or transient, of the symptoms or, adverse effects of a condition, such as, for example, reduction of adverse effects associated with or that occur upon administration of an NaPi2b-targeted polymer antibody-drug conjugate.

As used herein, when referencing dosage based on “body surface area” (BSA; m²) is the measured or calculated surface area of a human body. For many clinical purposes BSA is a better indicator of metabolic mass than body weight because it is less affected by abnormal adipose mass. Various calculations have been published to arrive at the BSA without direct measurement. In the following formulae, BSA is in m², W is mass in kg, and H is height in cm. The most widely used is the Du Bois, Du Bois formula: BSA=0.007184×W^{0.425}×H^{0.725}. Other methods of determining BSA include for example, the Mosteller, Haycock, Gehan and George, Boyd, Fujimoto, Takahira, Shuter and Aslani or Schlich formulas.

As used herein, “treating” or “treat” describes the management and care of a patient for the purpose of combating a disease, condition, or disorder and includes the administration of a conjugate of the disclosure, or a pharmaceutical composition thereof in combination with an immunomodulatory therapy, e.g., an immuno-oncology agent such as an immune checkpoint inhibitor, to alleviate the symptoms or complications of a disease, condition or disorder, or to eliminate the disease, condition or disorder.

As used herein, “prevention” or “prophylaxis” refers to reduction in the risk of developing a disease or condition, or reduction or elimination of the onset of the symptoms or complications of the disease, condition or disorder.

The term “effective amount” or “sufficient amount”, as it refers to an active agent, refers to the amount necessary to elicit the desired biological response. As used herein, a “therapeutically effective amount” or a “therapeutically effective dose” refers to an amount or quantity of an agent, compound, material, or composition containing a compound that is at least sufficient to produce a detectable therapeutic effect. The effect can be detected by any assay method known in the art. The precise effective amount for a subject will depend upon the subject's body weight, size, and health; the nature and extent of the condition; and the therapeutic selected for administration.

A “subject” includes a mammal. The mammal can be e.g., any mammal, e.g., a human, primate, bird, mouse, rat, fowl, dog, cat, cow, horse, goat, camel, sheep or a pig. Preferably, the mammal is a human.

As used herein, “unit dose form” or “unit dosage form” refers to physically discrete units suitable for human and animal subjects and packaged individually as is known in the art.

As used herein, a single dosage formulation refers to a formulation as a single dose.

As used herein, “temporal proximity” refers to that administration of one therapeutic agent (e.g., a NaPi2b-targeted polymer antibody-drug conjugate disclosed herein) occurs within a time period before or after the administration of another therapeutic agent (e.g., an immune checkpoint inhibitor disclosed herein), such that the therapeutic effect of the one therapeutic agent overlaps with the therapeutic effect of the other therapeutic agent. In some embodiments, the therapeutic effect of the one therapeutic agent completely overlaps with the therapeutic effect of the another therapeutic agent. In some embodiments, “temporal proximity” means that administration of one therapeutic agent occurs within a time period before or after the administration of another therapeutic agent, such that there is a synergistic effect between the one therapeutic agent and the another therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

As used herein a “kit” refers to a combination of components, such as a combination of the compositions herein and another item for a purpose including, but not limited to, reconstitution, activation and instruments/devices for delivery, administration, diagnosis and assessment of a biological activity or property. Kits optionally include instructions of use.

As used herein “platinum-sensitive recurrent disease” refers to a subject having achieved either a partial or complete response to 4 or more cycles in their penultimate platinum-containing regimen and progression of their disease >6 months after completion of the last dose of platinum containing therapy in the penultimate regimen.

As used herein “recurrent disease” refers to a subject having disease progression following partial or complete response to one or more therapeutics. In some embodiments, the recurrence can be local to the original site of disease (i.e. one or more ovaries) or at a distal or metastatic location.

As used herein “platinum-sensitive cancer” refers to a cancer that responds to treatment with anticancer drugs that contain the metal platinum, such as cisplatin and carboplatin. Cancers that respond to treatment but then come back after a certain period may also be considered platinum sensitive. For example, ovarian cancer that comes back 6 or more months after platinum-based treatment is considered platinum sensitive. Knowing whether cancer is platinum sensitive may help plan further treatment.

As used herein, the term “carboplatin” means a platinum co-ordination compound, diammine [1,1-cyclobutanedicarboxylato(2-)-0, 0′]. It is available commercially in various forms, such as, for example, a lyophilized powder or a pre-concentrate aqueous solution.

As used herein the term “Area under the Curve” or “AUC” defines the dose of carboplatin administered to a subject. In some aspects, the AUC is defined as the area under the plasma concentration/time curve. In some aspects, the AUC is expressed as mg/ml/min.

As used herein the term “maintenance therapy” refers to continuation of treatment beyond standard chemotherapy. In some aspects, standard chemotherapy can include a platinum-containing agent such as carboplatin. In some aspects, the continuation of treatment comprises the administration of an additional therapeutic agent. In some aspects, the additional therapeutic agent includes, but is not limited to, PARP inhibitors, such as, for example, olaparib and niraparib following front-line ovarian cancer chemotherapy, PARP inhibitors, such as, for example, olaparib, niraparib and rucaparib following recurrent ovarian cancer chemotherapy in patients with platinum-sensitive disease and bevacizumab following chemotherapy plus bevacizumab in front line and platinum-sensitive recurrent ovarian cancer. In a preferred aspect, the additional therapeutic used in the maintenance therapy comprises a NaPi2b-targeted antibody polymer-drug conjugate of the disclosure.

As used herein the term “progression-free survival” refers to the length of time during and after the treatment of a disease, such as cancer, and more specifically ovarian cancer, that a patient lives with the disease without progression of the disease.

The present disclosure is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

The present disclosure is intended to include all isomers of the compound, which refers to and includes, optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers.

### OTHER EMBODIMENTS

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following examples are illustrative and are not intended to be limiting and it will be readily understood by one of skill in the art that other reagents or methods may be utilized.

### ABBREVIATIONS

The following abbreviations are used in the reaction schemes and synthetic examples, which follow. This list is not meant to be an all-inclusive list of abbreviations used in the application as additional standard abbreviations, which are readily understood by those skilled in the art of organic synthesis, can also be used in the synthetic schemes and examples.

| Abbreviation | Full Term |
|---|---|
| ADC | antibody drug conjugate |
| AE | adverse event |
| AF-HPA | Auristatin F-hydroxypropyl amide |
| ALT | alanine aminotransferase |
| AST | aspartate aminotransferase |
| AUC | area under the curve |
| BRCA | Breast Cancer gene |
| BSA | body surface area |
| CDRH | Center for Devices and Radiological Health |
| CDx | Companion/complementary Diagnostic |
| CI | Confidence Interval |
| Cmax | Maximum concentration |
| CR | complete response |
| DAR | drug-to-antibody ratio |
| DCR | disease control rate |
| DES | dose-escalation |
| DLT | dose limiting toxicities |
| DOR | duration of response |
| ECOG | Eastern Cooperative Oncology Group |
| HGSOC | high-grade serous ovarian cancer |
| HNSTD | highest non-severely toxic dose |
| ICH | International Conference on Harmonization |
| IHC | immunohistochemistry |
| IV | intravenous(ly) |
| m2 | square meter |
| mAb | monoclonal antibody |
| MedDRA | Medical Dictionary for Regulatory Activities, Version |
| | 20.1 |
| NaPi2b | type II sodium-dependent phosphate transporter |
| | (SLC34A2) |
| NE | not evaluable for response per RECIST v1.1 |
| NSAID | nonsteroidal anti-inflammatory drug |
| NSCLC | non-small cell lung cancer |
| OC | Ovarian cancer |
| ORR | objective response rate |
| PK | pharmacokinetic(s) |
| Pap | papillary renal |
| PARPi | poly ADP ribose polymerase inhibitor |
| PFS | progression-free survival |
| PFS2 | progression-free survival 2 |
| PR | partial response |
| PP | per protocol |
| Q | Every |
| QTc | corrected QT interval |
| RECIST v1.1 | Response Evaluation Criteria in Solid Tumors, version |
| | 1.1 |
| SAE | serious adverse event |
| SAP | statistical analysis plan |
| SD | stable disease |
| TFST | time to first subsequent therapy |
| TPS | tumor proportion score |
| ULN | upper limit of normal |
| Vss | steady state volume of distribution |

### General Information

XMT-1536 was prepared as described in U.S. patent Ser. No. 10/947,317(B2).

AF-HPA was prepared as described in U.S. Pat. No. 8,808,679(B2)

CDRs were identified by the Kabat numbering scheme.

### Example 1: Study Design and Rationale

The study presented herein is a double-blind, randomized, placebo controlled (2:1 XMT-1536: placebo), Phase 3 study in patients with recurrent, platinum-sensitive high-grade serous ovarian cancer (HGSOC) including fallopian tube and primary peritoneal cancer in the maintenance setting. Participants must have had 4 to 8 cycles of platinum-based chemotherapy in their most recent treatment regimen, including carboplatin or cisplatin±paclitaxel, docetaxel, pegylated liposomal doxorubicin or gemcitabine in the 2nd-4th line setting for the treatment of platinum-sensitive recurrent disease, with no evidence of disease (NED)/complete response (CR)/partial response (PR)/ or stable disease (SD) as best response.

The standard of care for patients with platinum-sensitive recurrent HGSOC consists of platinum-based chemotherapy in the United States. The majority of these patients will have received bevacizumab, a PARPi, or a combination of the two as maintenance therapy following standard front-line platinum-based chemotherapy. The PARPi niraparib is approved for maintenance treatment in those who have attained a CR or PR to front-line therapy and bevacizumab is approved for patients with stages III and IV disease following initial surgical resection in combination with carboplatin and paclitaxel, followed by single agent therapy. The combination of the PARPi olaparib and bevacizumab is approved for maintenance therapy (following bevacizumab combined with chemotherapy) in patients whose cancers are associated with homologous recombination (HR) deficiency status defined by either a deleterious or suspected deleterious BRCA mutation, and/or genomic instability.

For those who have not received a PARPi as maintenance following front-line chemotherapy, maintenance therapy with niraparib, rucaparib and olaparib is approved in those with platinum-sensitive recurrent HGSOC with CR or PR as best response to the most immediate platinum-based regimen. In general, the clinical benefit for PARPi's is enriched for patients with either germline or somatic BRCA mutations. This is consistent with real-world evidence for lack of PARPi utilization in patients without either a germline or somatic BRCA mutation (BRCA wild-type). In an analysis of real-world data on maintenance treatment uptake, an estimated 63% of women with BRCA mutations receive maintenance treatment, compared to 37% of women without BRCA mutations. Therefore, eligibility for the current Phase 3 study only requires prior PARPi for participants with a known germline or somatic BRCA mutation.

This study excludes participation for those with only one prior line of platinum-based therapy since maintenance treatment with PARP inhibitors and/or bevacizumab is considered standard in this context. The study also excludes participation for those with more than 4 prior lines of platinum-based therapy given the rarity of this scenario.

In addition to the requirement related to prior maintenance therapy with a PARPi, this study excludes participants receiving bevacizumab in combination with their most recent platinum-based regimen; and would continue to receive bevacizumab in their maintenance therapy. The primary objective of demonstrating superiority of an investigational agent compared to monitoring/placebo in maintenance is consistent within the standard for clinical trials in this population and can be justified in patients who have already received standard maintenance regimens (thus with unmet medical need).

The median PFS for PARPi naïve patients following CR or PR to 2^{nd} or 3^{rd} line platinum-based combination chemotherapy for platinum-sensitive recurrent disease is only approximately 4.8 months, based on review of data from NOVA, ARIEL3 and Study 19. Thus, an effective maintenance treatment following completion of platinum-based chemotherapy in patients who have already received standard maintenance regimens would address this unmet medical need.

The inclusion of participants who have experienced CR or PR is consistent with clinical trials evaluating PARPi therapy in the maintenance setting. The inclusion of those with stable disease as best response to their most recent platinum-based regimen is unique with no currently approved indication for maintenance therapy; however, the inclusion of participants with stable disease in this case is consistent with the mechanism of action for XMT-1536 and known anti-tumor activity as a single agent in patients with platinum-resistant ovarian cancer, and at the same time addresses the unmet medical need for this specific patient sub-population which in all likelihood experiences a worse prognosis than the sub-population with either a CR or PR.

A non-balanced randomization ratio of 2:1 for the experimental arm over the control arm balances the principle of equipoise in clinical trial research with acceptability to prospective study participants. This randomization ratio has been utilized in other ovarian cancer maintenance trials (e.g. SOLO1, PRIMA, NOVA, PAOLA-1).

Randomization of participants will be stratified based on their response to last platinum-based regimen (NED or CR versus PR versus SD), number of prior lines of platinum-based therapy (2 versus 3 or 4) and previous treatment with a PARP inhibitor treatment (yes or no). Such stratification safeguards against potential imbalances in these factors which are well known to be associated with therapeutic benefit to a variety of agents and with prognosis and thus may impact the primary endpoint. The primary objective of demonstrating superiority of XMT-1536 with respect to PFS compared to placebo in maintenance is consistent with the standard for clinical trials in this population. The treatment effect size represented by a hazard ratio for disease progression of 0.60 is based on the premise of clinically meaningful benefit in this population at high risk of early disease progression which includes patients with stable disease as best response to their most recent platinum-based regimen and receipt of prior standard maintenance therapies. This hazard ratio translates into a 3.2-month prolongation of PFS for the experimental over the placebo group. In order to capture early progression events as accurately as possible, radiologic disease assessments for the first 9 months will be performed at 6-week intervals. The primary endpoint of PFS ascertained by BICR using RECIST v1.1 has been utilized as a primary endpoint in a number of ovarian cancer phase 3 studies evaluating PARP inhibitors in the maintenance setting, including PRIMA. The use of BICR also provides greater objectivity than investigator determined assessment of PFS. In addition to the inclusion of a placebo control arm, use of Blinded Independent Central Review (BICR) is warranted to further limit bias for both investigators and patients with respect to PFS.

The participant's disease must be positive for NaPi2B expression, as measured by central laboratory (archived or recent biopsy). Based on the preliminary data from the ongoing MER-XMT-1536-1 clinical trial, XMT-1536 demonstrated greater likelihood of clinical activity in these patients, with an ORR of 32% and DCR of 74% in patients with higher NaPi2b expression (n=47) with a median duration of response of approximately 5 months. For this study, a tumor proportion score (TPS) of ≥75 by immunohistochemistry (IHC) will be considered positive (high) and a TPS of <75 will be considered negative (low). This cut-off was selected based on the ongoing training and validation assay work in the MER-XMT-1536-1 study.

The starting dose selected for this study, 30 mg/m² (capped at BSA 2.2 m2) q4wk, is informed by the dose escalation portion of the MER-XMT-1536-1 monotherapy trial in multiple solid tumors, and preliminary data from the dose expansion portion of that study in patients with high grade serous ovarian cancer (HGSOC) and non-small cell lung cancer (NSCLC) and led to the selection of 30 mg/m² with BSA capped at 2.2 mg/m², administered every 28 days (same schedule as for MER-XMT-1536-1).

Participants will be treated with XMT-1536 until disease progression, unacceptable toxicity, voluntary discontinuation, death, or a treatment duration of 18 months, whichever comes first. The 18 months maximum treatment duration is intended to balance the potential for chronic toxicity/late adverse effects, acceptability to participants, relative to the therapeutic benefit beyond this time-point. Participants may be treated beyond 18 months, if in the opinion of the treating physician, they may derive further benefit from continuous treatment, following consultation with the Medical Monitor.

For this study, the EQ-5D-5L, EORTC-QLQ, OV28, EORTC-QLQ-C30, and FOSI will be utilized to investigate the effect of maintenance treatment with XMT-1536 versus placebo on quality of life (QoL). Taken together, these assessment tools will holistically inform on overall well-being, symptoms related to the disease under study, and the effect of study drug related toxicities as they related to QoL. These questionnaires are described more fully in the protocol.

### Primary Objective:

Demonstrate superiority in Progression-free Survival (PFS) as assessed by Blinded Independent Central Review (BICR) using Response Evaluation Criteria in Solid Tumors (RECIST) Version 1.1 of XMT-1536 versus placebo as maintenance therapy

### Key Secondary Objective:

Compare Overall Survival (OS) of XMT-1536 versus placebo as maintenance therapy

### Other

### Secondary Objectives:

Compare PFS as assessed by Investigator using RECIST v1.1 of XMT-1536 versus placebo as maintenance therapy.

Compare the Objective Response Rate (ORR) as assessed by Investigator using RECIST v1.1 of XMT-1536 versus placebo

Evaluate safety and tolerability in participants treated with XMT-1536 versus placebo as maintenance therapy.

### Exploratory Objectives

Compare Progression-free survival 2 (PFS2) as assessed by Investigator using RECIST v1.1 of XMT-1536 versus placebo as maintenance therapy

Compare time to first subsequent therapy (TFST) as assessed by Investigator using RECIST v1.1 of XMT-1536 versus placebo as maintenance therapy

Evaluate additional measures of clinical benefit for XMT-1536 versus placebo as maintenance treatment using patient-reported outcomes (PROs)

Assess the population pharmacokinetics of XMT-1536

Assess development of anti-drug antibody (ADA) and neutralizing antibody (nAb) in response to XMT-1536 exposure

Assess the population pharmacokinetics and exposure/response of XMT-1536, including exploration of the relationship between exposure and efficacy (ORR, DOR, and PFS) and safety endpoints.

Evaluate the association of objective response with tumor expression of genes other than NaPi2b or other tumor molecular and histologic features.

### Number of Participants

The study plans to enroll 350 participants.

### Eligibility

To be eligible for enrolment in this study, all participants must fulfil all the inclusion criteria and none of the exclusion criteria as defined below:

### Diagnosis and Main Criteria for Inclusion:

1. Participants must be at least 18 years of age, and female.
2. Participant must have an ECOG performance status 0 or 1
3. Participant must have a histological diagnosis of high grade serous ovarian cancer, which includes fallopian tube and primary peritoneal cancer, that is metastatic or recurrent.
4. Participant must be able to understand the study procedures and agree to participate in the study by providing written informed consent.
5. Participant must have platinum-sensitive recurrent disease, defined as having achieved either a partial or complete response to 4 or more cycles in their penultimate platinum-containing regimen and their disease progressing more than 6 months after completion of the last dose of platinum containing therapy in the penultimate regimen.
6. Participant must have had 4 to 8 cycles of platinum-based chemotherapy in 2^{nd} to 4^{th} line setting in their most recent treatment regimen as defined below:
   Platinum-based chemotherapy regimens allowed immediately preceding enrollment to the study are: carboplatin or cisplatin±: paclitaxel, docetaxel, pegylated liposomal doxorubicin or gemcitabine
   Participant must receive first study treatment infusion between 4 and 12 weeks after completing final dose of platinum in the most recent platinum-based regimen.
   Definitions for prior lines of therapy:
      Adjuvant±neoadjuvant considered one line of therapy as long as they are the same regimens (e.g., platinum/taxane for 3 cycles before surgery followed by platinum/taxane for 3 cycles after surgery)
      Maintenance therapy (e.g., bevacizumab, PARPi, endocrine therapy) will be considered as part of the preceding line of therapy (i.e., not counted independently)
      Therapy given for only 1 cycle and discontinued due to toxicity in the absence of progression will not be counted as a new line of therapy; therapy given for 2 or more cycles will be counted as a line of therapy. Substitutions of different platinum agents or taxanes will not be counted as new lines.
      Hormonal therapy (e.g., tamoxifen, letrozole) will be counted as a separate line of therapy unless given as maintenance.
7. Participant must have had as their best response to last line of treatment one of the following: No Evidence of Disease (NED); Complete Response (CR); Partial Response (PR); OR Stable Disease (SD), defined as follows:

| | |
|---|---|
| NED | Initiation of most recent platinum-based regimen following |
| | surgical resection, with no evidence of radiographically |
| | measurable or evaluable disease at initiation of most |
| | recent platinum-based regimen and at screening |
| CR | Complete response as defined by RECIST version 1.1 |
| PR | Partial response as defined by RECIST version 1.1 |
| SD | Stable disease as per RECIST v1.1 and with no increase in |
| | radiographically measurable or evaluable disease, comparing |
| | most recent previous imaging performed more than 28 days |
| | prior to screening study to screening study. |
| | Absence of >15% rise in CA-125 levels when comparing |
| | screening to most recent prior value performed at least |
| | 7 days before the screening value. This criterion does not |
| | apply to cases where both values are within normal range. |

Participants with NED, CR, or PR as their best response to most recent line of treatment and who have not received treatment with a prior PARP inhibitor must have definitive BRCA1 and BRCA2 testing results that demonstrate no evidence of a deleterious BRCA1 or BRCA2 mutation. Somatic BRCA mutation testing is required for participants who are classified as not having a deleterious mutation by germline testing alone.
9. Participant must provide either an archival tumor tissue block or fresh cut slides at screening for measurement of NaPi2b expression by a central laboratory. If sufficient archival tumor tissue is not available, then a tumor tissue block or slides must be obtained from a fresh biopsy and provided to the central laboratory. Confirmation of a NaPi2b-H/positive tumor by the central laboratory is required prior to randomization.
10. Participants with toxicity from prior therapy or surgical procedures must have recovered to ≤Grade 1. Participants with alopecia, stable immune-related toxicity such as hypothyroidism on hormone replacement, adrenal insufficiency treated with ≤10 mg daily prednisone (or equivalent), or chronic Grade 2 peripheral sensory neuropathy after prior taxane therapy is an exception to this criterion and may qualify for this study.
11. Participants must have cardiac left ventricular ejection fraction (LVEF) ≥50% or ≥the institution's lower limit of normal as measured by either Echo or WUGA scan
12. Participants must have adequate organ function within 14 days prior to enrollment as defined by the following criteria:

| | |
|---|---|
| Absolute neutrophil | ≥1500 cells/mm³ |
| count (ANC), | |
| Platelet count | ≥100,000/mm³ |
| Hemoglobin | ≥9 g/dL |
| INR, activated partial | In participants not on anticoagulation |
| thromboplastin time | therapy: INR, activated partial |
| (aPTT), and | thromboplastin time (aPTT), and |
| prothrombin time (PT) | prothrombin time (PT) all within |
| | 1.2 times the institutional upper limit |
| | to normal (ULN). Patients on |
| | anticoagulation therapy are allowed if |
| | their relevant laboratory values are |
| | within the therapeutic window. |
| Estimated glomerular | ≥45 mL/min according to CKD-EPI |
| filtration rate (GFR). | Creatinine Equation or institutional |
| | standard method. |
| Total bilirubin | ≤ULN |
| | Note: Participants with asymptomatic |
| | elevations in unconjugated bilirubin |
| | due to Gilbert syndrome or stable |
| | chronic hemolytic anemia (e.g., |
| | hereditary spherocytosis, sickle cell |
| | disease, thalassemia intermedia) may |
| | be eligible after discussion with the |
| | Sponsor Medical Monitor. |
| Aspartate aminotransferase | ≤1.5 times the institutional ULN |
| (AST or SGOT) and alanine | |
| aminotransferase (ALT or SGPT). | |

13. During the study female study participants of child-bearing potential (WOCBP) must use a contraceptive method that is highly effective during study treatment and for at least 6 months after the last dose of study treatment.
   The Investigator is responsible for review of medical history, menstrual history, and recent sexual activity to decrease the risk for inclusion of a woman with an early undetected pregnancy.

### Exclusion Criteria

Participant will not be eligible for study entry if any of the following criteria are met:
1. Participant has received prior treatment with mirvetuximab soravtansine or another ADC containing an auristatin or maytansinoid payload.
2. Participant has received bevacizumab in combination with last platinum-based regimen or plans to receive maintenance therapy outside the study intervention.
3. Participant has clinical signs or symptoms of gastrointestinal obstruction and/or requirement for parenteral hydration or nutrition.
4. Participant has ascites or pleural effusion managed with therapeutic paracentesis or thoracentesis within 28 days prior to signing the principal study consent form.
5. Participant has history of cirrhosis, hepatic fibrosis, esophageal or gastric varices, or other clinically significant liver disease. Testing beyond laboratory studies otherwise defined in the eligibility criteria, to diagnose potentially clinically significant liver disease based on risk factors such as hepatic steatosis or history of excessive alcohol intake, will be based on clinical judgement of the investigator.
6. Participants cannot receive drugs associated with hepatotoxicity concurrent with XMT-1536 administration.
7. Participant currently uses or intermittent supplementary oxygen therapy.
8. Participant has history of or suspected pneumonitis or interstitial lung disease.
9. Participant has oxygen saturation on room air <93%.
10. Participant has had major surgery or systemic anti-cancer therapy within 28 days of starting study treatment.
11. Participant has a low-grade, clear cell, endometrioid, mucinous, carcinosarcoma, germ-cell, mixed histology, or stromal tumor.
12. Participant has untreated CNS metastases (including new and progressive brain metastases), history of leptomeningeal metastasis, or carcinomatous meningitis.
   a. Participants are eligible if CNS metastases are adequately treated and are neurologically stable for at least 2 weeks prior to enrollment.
   b. In addition, participants must be either off corticosteroids, or on a stable/decreasing dose of ≤10 mg daily prednisone (or equivalent) prior to first dose of study treatment. Anticonvulsants are allowed except for those drugs associated with liver toxicity (see full protocol).
13. Participant has untreated, known human immunodeficiency virus (HIV), hepatitis B virus (HBV), or hepatitis C virus (HCV). In addition, negative serology is required during screening (baseline) for HBV and HCV:
   HBV: Participants with serologic evidence of chronic HBV infection should have an HBV viral load below the limit of quantification to be eligible.
   HCV: Participants with a history of HCV infection should have completed curative antiviral treatment and HCV viral load below the limit of quantification.
   Screening for HIV is not required except if mandated by local regulations or indicated based on clinical assessment.
14. Participant has current severe, uncontrolled systemic disease (e.g., clinically significant cardiovascular, pulmonary, or metabolic disease) or intercurrent illness that could interfere with per-protocol evaluations. Further, participants are excluded with the following characteristics:
   A marked baseline prolongation of QTcF interval CTCAE Grade>1: repeated demonstration of a QTc interval >480 milliseconds (ms) using Fridericia's QT correction formula.
   A history of additional risk factors for Torsades de Pointes (e.g., heart failure, hypokalemia, family history of Long QT Syndrome).
15. Has a diagnosis of additional malignancy that required treatment within 2 years prior to screening, except for adequately treated basal cell or squamous cell skin cancer, or carcinoma in situ of the breast or of the cervix
16. Participant has clinically significant corneal disease.
17. Participant is unwilling to be transfused with blood components.
18. Participant is receiving concurrent anti-cancer therapy (e.g. chemotherapy, radiation therapy, biologic therapy, immunotherapy, hormonal therapy, investigational therapy).
19. Participant is unable or unlikely to comply with dosing schedule and study evaluations.
20. Participant is using strong CYP450 3A4 inhibitors or inducers that cannot be discontinued while receiving study treatment.
21. Participants who are pregnant or nursing. For WOCBP, Pregnancy status must be confirmed with a negative highly sensitive pregnancy test (urine or serum as required by local regulations) within 72 hours before the first dose of study treatment.

### General Considerations

This study consists of a Pre-Screening Period to allow for central confirmation of NaPi2b expression levels (duration dependent upon availability of tissue), a Screening Period (Day −28 to Day −1), a Treatment Period, an End of Treatment (EOT) Visit (+7 days), a Safety Follow-up Visit (60 days [±7] after last dose), and Overall Survival follow-up (via telephone) every 90 (±14) days until death or the end of study data collection. In order to confirm NaPi2b expression, each participant must submit either an archival tumor tissue block or fresh cut slides to a central laboratory; results must be confirmed prior to randomization. During the Treatment Period, study drug administration will occur in 4-week cycles. Participants will receive XMT-1536 or placebo q4 weeks until progressive disease (PD), unacceptable toxicity, withdrawal of consent, Investigator's decision, death, or for up to a total of 18 months, whichever comes first. Participants may be treated beyond 18 months, if in the opinion of the treating physician, they may derive further benefit from continued treatment, following discussion with the Medical Monitor.

Participants must have a baseline tumor assessment (computed tomography [CT] or magnetic resonance imaging [MRI]) of the chest, abdomen, pelvis, and other sites as clinically indicated within 28 days prior to the first dose of study drug. Tumor imaging will be repeated every 6 weeks (±3 days) for the first 12 months of treatment, then every 12 weeks (±7 days) until disease progression or initiation of non-study anti-cancer therapy (following discontinuation of study treatment), whichever comes sooner. The same imaging technique should be used throughout the study.

CA-125 testing to assess extent of disease will be conducted as outlined in the SoA and at any time when progression of disease is suspected.

All adverse events (AEs) will be collected and recorded for each participant from the first dose of study drug treatment through the Follow-up Period or until alternate anticancer treatment has been initiated, whichever occurs earlier; any pregnancies that occur within 180 days post-treatment are to be reported. All Serious Adverse Events (SAEs) will be collected and recorded for each participant from the signing of the main informed consent through the Follow-up Period (or until alternate anticancer treatment has been initiated; whichever occurs earlier). All AEs and SAEs experienced by a participant, regardless of the suspected causality, will be monitored until the AE or SAE has resolved, until abnormal laboratory values have returned to baseline or normalized, until there is a satisfactory explanation for the changes observed, until the participant is lost to follow-up or withdraws consent, or until the participant has died.

### Study Committees

A Data Safety Monitoring Board (DSMB) will be established to provide independent review and assessment of the safety data in a systematic manner and to safeguard the interest and safety of the participants in the study. The DSMB makeup and timeframe for reviews will be outlined in the DSMB charter. In general, the DSMB will be tasked with making a recommendation to the Sponsor to continue, modify, or stop the study based on their assessment of safety information.

Additionally, a Blinded Independent Central Review process will be in place to support the primary objective of this study, demonstration of superiority in PFS of upifitamab rilsodotin versus placebo as maintenance therapy as assessed by BICR using RECIST V1.1. Upon notification by Sponsor, sites will submit all imaging and supportive clinical data for central radiologic assessment by 2 independent radiologists, and an arbiter, if necessary. This process will be documented in detail in the imaging charter.

### Patient-Reported Outcomes

The EQ-5D-5L, EORTC-QLQ-OV28, EORTC-QLQ-C30, and FOSI will be collected in conjunction with tumor assessments, as outlined in the SOA, while the participant is receiving study treatment.

### Pharmacokinetics and ADA

Plasma samples for PK determination as well as antidrug antibody (ADA) assessment will be collected during study treatment as outlined in the Schedule of Activities. Area under the concentration-time curves (AUCs) will be derived based on the results of plasma PK sample analysis. Results of 3-tier ADA assays (screening, confirmation and titer) and competitive ligand binding assay as neutralizing antibody assay (NAb) will be correlated with clinical activity, PK, as well as safety assessments.

### Criteria for Evaluation:

Below is a listing of the criteria for evaluation of a participant Safety
The frequency and grade of AEs based on CTCAE Version 5.0
Changes in clinical laboratory parameters vital signs, ECOG performance status, ECG parameters, physical examinations, and usage of concomitant medications

### Clinical Activity

Antineoplastic activity of XMT-1536
   PFS based on BICR using RECIST v1.1
   PFS based on Investigator assessment using RECIST v1.1
   ORR using RECISTv1.1
   OS, as measured from the date of first dose to the date of death by any cause
   PFS2 based on Investigator assessment using RECIST v1.1
   TFST

### Patient-Reported Outcomes

Observed changes from baseline in the following PROs:
   EQ-5D-5L
   EORTC-QLQ-OV28
   EORTC QLQ-C30
   FOSI

### Pharmacokinetics and Antidrug Antibodies

Pharmacokinetics of XMT-1536 (Cₘₐₓ, C_{trough}, tₘₐₓ, AUC, t_{1/2}, CL, and Vₛₛ)
Effect of XMT-1536 exposure of safety and efficacy endpoints, as outlined in the specific SAP
Immunogenicity: Plasma samples for analysis of XMT-1536 neutralizing antibodies.

### Statistical Methods:

### Sample Size Consideration

The study was designed to detect a hazard ratio of 0.60, translating in a 67% improvement in median PFS from 4.8 months in the placebo arm to 8 months in the XMT-1536 arm.

Approximately 175 events (i.e., disease progression or death) provides 90% power to detect a hazard ratio of 0.6 with an overall 2-sided type 1 error rate of 0.05.

Approximately 350 patients will be randomized to the two treatment arms in a 2:1 (XMT-1536: Placebo) ratio. Accrual of participants is expected to take approximately 18 months, with the last patient followed for 12 months resulting in a total study duration of 30 months. A common exponential drop-out rate of 9.5% is assumed.

### Analysis Populations

Intent-to-Treat: All randomized participants
Per Protocol: All dosed participants who satisfy all inclusion and none of the exclusion criteria and who do not have major protocol deviations which impact the primary efficacy assessment. The list of major protocol deviations that exclude patients from the PP population will be provided in the Statistical Analysis Plan (SAP).
Safety: All subjects who received any amount of study treatment.
PROs: Patients with a minimum of an evaluable score at baseline and at least one evaluable follow-up form.
PK: All participants with at least one post-infusion sample
ADA: All participants who receive at least 1 dose of study drug, have provided the pre-treatment blood sample and at least 1 post-treatment plasma sample at or after 96 hours.

### General Methods

Descriptive statistics will be used to display the results. Continuous variables, including baseline characteristics, will be summarized by reporting the number of observations, mean, standard deviation, median, minimum and maximum. Categorical/discrete variables will be summarized using frequency tables showing the number and percentage of participants within a category.

### Efficacy Analysis

The primary efficacy analysis will compare the distributions of PFS based on BICR between the 2 arms using a two-sided log-rank test, stratified by response to last platinum-based regimen (NED/CR vs PR vs SD), number of prior lines of platinum-based therapy (2 vs 3/4) and previous treatment with a PARPi (Yes vs. No). The PFS curves, median PFS and PFS rates at 6, 9 and 12 months for each randomized arm will be estimated using the Kaplan-Meier method. Corresponding two-sided 95% confidence intervals will also be provided. The hazard ratio will be estimated in a Cox proportional hazards model using randomized treatment arm and the above stratification factors as covariates.

A hierarchical hypothesis testing approach will be employed where the primary endpoint, PFS as assessed by BICR, is tested at the 2-sided alpha level of 0.05. If PFS based on BICR is found to be significant, then OS will be tested

An interim analysis for OS will be performed at the time of the PFS by BICR analysis. The OS efficacy boundaries at the interim and final analyses will be derived based on the number of deaths using a Lan-DeMets O'Brien-Fleming approximation spending function

Analysis of PFS based on investigator assessment will be conducted as a sensitivity analysis to the primary endpoint.

PFS based on investigator assessment and OS will be analyzed in a similar manner as described above for PFS based on BICR.

ORR will be compared between the 2 arms using the Cochran-Mantel-Haenszel test, controlling for the 3 stratification factors.

Additional exploratory endpoints will be analyzed as outlined in the Statistical Analysis Plan.

### Safety Analysis

All participants who receive at least one dose of study treatment will be evaluated for safety. The incidence rates of treatment emergent adverse events, treatment related adverse events, serious treatment emergent adverse events (SAEs) and adverse events of clinical interest (AECIs) will be summarized by MedDRA preferred terms and system organ class (SOC). The frequency of occurrence of overall toxicity, categorized by the maximum toxicity grades (severity) and maximum relationship to study treatment will also be described.

Listings of laboratory test results and CTCAE grades will be generated, and descriptive statistics summarizing the changes in laboratory tests over time will be presented. Additionally, the prevalence and incidence of ADA and nAB levels will be reported.

### Patient-Reported Outcomes

The EQ-5D-5L, EORTC-QLQ OV28, EORTC-QLQ-C30, FOSI will be utilized to investigate the effect of maintenance treatment with XMT-1536 versus placebo on quality of life (QoL). These assessment tools will be used to describe overall well-being, symptoms related to the disease under study, and the effect of study drug related toxicities as they related to QoL.

### PK and ADA Analysis

Plasma concentrations, PK parameters, and ADA data will be summarized with descriptive statistics. Blood samples for XMT-1536 will be collected at the timepoints outlined in the Schedule of Activities with sparse PK sampling. PK and exposure parameters based on the population PK model, defined in the PK SAP, will be estimated.

### OTHER EMBODIMENTS

While the invention has been described in conjunction with the detailed description thereof, the foregoing description is intended to illustrate and not limit the scope of the invention, which is defined by the scope of the appended claims. Other embodiments, advantages, and modifications are within the scope of the following claims.

1. A method of treating ovarian cancer in a subject having a recurrent, platinum-sensitive ovarian cancer, comprising administering to the subject a NaPi2b-targeted antibody polymer-drug conjugate by infusion at a dose of between about 20 mg/m² to about 36 mg/m² on the first day of treatment and every four weeks thereafter,
wherein the NaPi2b-targeted antibody polymer-drug conjugate is: 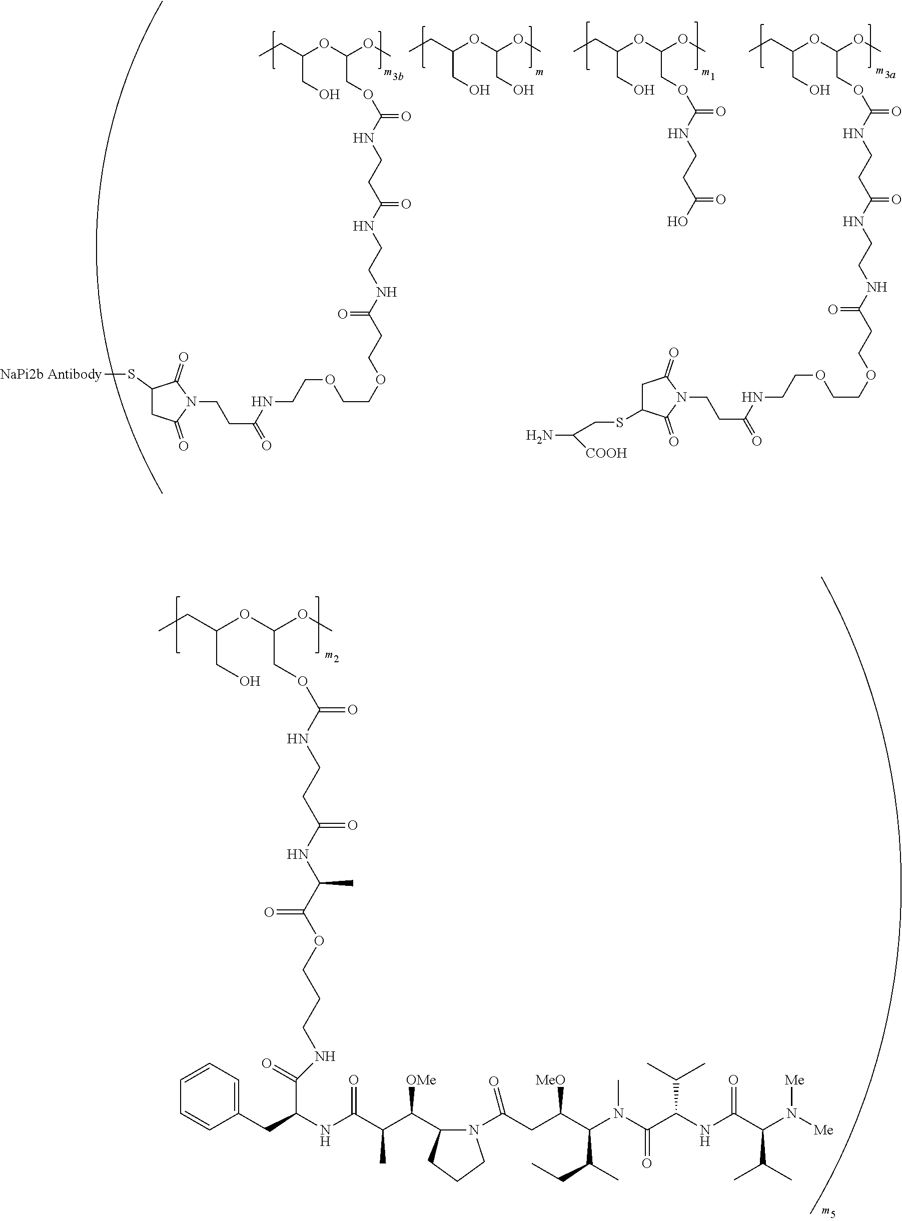
wherein:
the polymer-drug conjugate comprises a polymeric scaffold comprising poly(1-hydroxymethylethylene hydroxymethyl-formal) (PHF), wherein the PHF has a molecular weight ranging from 5 kDa to 10 kDa;
m is an integer from 20 to 75,
m₁ is an integer from about 5 to about 35,
m₂ is an integer from about 3 to about 10,
m₃ₐ is an integer from 0 to about 4,
m_{3b} is an integer from 1 to about 5,
the sum of m, m₁, m₂, m₃ₐ, and m_{3b} ranges from about 40 to about 75,
m₅ is an integer from about 2 to about 6, and
the NaPi2b-targeted antibody (XMT-1535) comprises a variable light chain complementarity determining region 1 (CDRL1) comprising the amino acid sequence SASQDIGNFLN (SEQ ID NO: 8); a variable light chain complementarity determining region 2 (CDRL2) comprising the amino acid sequence YTSSLYS (SEQ ID NO: 9); a variable light chain complementarity determining region 3 (CDRL3) comprising the amino acid sequence QQYSKLPLT (SEQ ID NO: 10); a variable heavy chain complementarity determining region 1 (CDRH1) comprising the amino acid sequence GYTFTGYNIH (SEQ ID NO: 5); a variable heavy chain complementarity determining region 2 (CDRH2) comprising the amino acid sequence AIYPGNGDTSYKQKFRG (SEQ ID NO: 6); and a variable heavy chain complementarity determining region 3 (CDRH3) comprising the amino acid sequence GETARATFAY (SEQ ID NO: 7).

2. The method of claim 1, wherein XMT-1535 comprises a variable heavy chain comprising the amino acid sequence of SEQ ID NO: 3 and a variable light chain comprising the amino acid sequence of SEQ ID NO: 4.

3. The method claim 1, wherein XMT-1535 comprises a heavy chain comprising the amino acid sequence of SEQ ID NO: 1 and a light chain comprising the amino acid sequence of SEQ ID NO: 2.

4. The method of claim 1, wherein the conjugate dose is about 20 mg/m².

5. The method of claim 1, wherein the conjugate dose is about 25 mg/m².

6. The method of claim 1, wherein the conjugate dose is about 30 mg/m².

7. The method of claim 1, wherein the conjugate dose is about 36 mg/m².

8. The method of claim 4, wherein the conjugate dose of about 20 mg/m² is capped at BSA 2.2 m².

9. The method of claim 5, wherein the conjugate dose of about 25 mg/m² is capped at BSA 2.2 m².

10. The method of claim 6, wherein the conjugate dose of about 30 mg/m² is capped at BSA 2.2 m².

11. The method of claim 7, wherein the conjugate dose of about 36 mg/m² is capped at BSA 2.2 m².

12. The method of claim 1, wherein the conjugate dose is administered at a maximum of about 80 mg.

13. The method of claim 1, wherein the ovarian cancer is high grade serous ovarian cancer.

14. The method of claim 13, wherein the high grade serous ovarian cancer is fallopian tube cancer or primary peritoneal cancer.

15. The method of claim 1, wherein the subject is administered the NaPi2b-targeted antibody polymer-drug conjugate on the first day of treatment and every four weeks thereafter for up to 18 cycles.

16. The method of claim 1, wherein the subject has received treatment with 4 to 8 cycles of platinum-based chemotherapy with no evidence of disease (ned)/complete response (cr)/partial response (pr)/ or stable disease (sd) as best response.

17. The method of claim 1, wherein the subject has stable disease as best response to their most recent platinum-based regimen.

18. The method of claim 1, wherein the subject experiences reduced and/or no progression of the ovarian cancer following treatment with the NaPi2b-targeted antibody polymer-drug conjugate relative to treatment with placebo.

19. The method of claim 1, wherein the subject experiences improved progression-free survival following treatment with the NaPi2b-targeted antibody polymer-drug conjugate relative to treatment with placebo.

20. The method of claim 1, wherein PHF has a molecular weight ranging from about 5 kDa to about 10 kDa, m is an integer from 30 to about 35, m₁ is an integer from 8 to about 10, m₂ is an integer from 2 to about 5, m₃ₐ is an integer from 0 to about 1, m_{3b} is an integer from 1 to about 2, the sum of m₃ₐ and m_{3b} ranges from 1 and about 4, and m₅ is an integer from about 3 to about 4.

21. The method of claim 1, wherein the ratio between m² and XMT-1535 is about 16:1 to 10:1.

22. The method of claim 19, wherein the ratio between m₂ and XMT-1535 is about 12:1 to 8:1.

23. The method of claim 19, wherein the ratio between m₂ and XMT-1535 is about 10:1.


SEQUENCE LISTING (15 sequences)

SEQ ID NO: 1 - protein, 449 aa, Homo sapiens
QVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 215 aa, Homo sapiens
DIQMTQSPSSLSASVGDRVTITCSASQDIGNFLNWYQQKPGKTVKVLIYYTSSLYSGVPSRFSGSGSGTDYTLTISSLQPEDFATYYCQQYSKLPLTFGQGTKLELKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 120 aa, Homo sapiens
QVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSSG

SEQ ID NO: 4 - protein, 108 aa, Homo sapiens
DIQMTQSPSSLSASVGDRVTITCSASQDIGNFLNWYQQKPGKTVKVLIYYTSSLYSGVPSRFSGSGSGTDYTLTISSLQPEDFATYYCQQYSKLPLTFGQGTKLELKR

SEQ ID NO: 5 - protein, 10 aa, Homo sapiens
GYTFTGYNIH

SEQ ID NO: 6 - protein, 17 aa, Homo sapiens
AIYPGNGDTSYKQKFRG

SEQ ID NO: 7 - protein, 10 aa, Homo sapiens
GETARATFAY

SEQ ID NO: 8 - protein, 11 aa, Homo sapiens
SASQDIGNFLN

SEQ ID NO: 9 - protein, 7 aa, Homo sapiens
YTSSLYS

SEQ ID NO: 10 - protein, 9 aa, Homo sapiens
QQYSKLPLT

SEQ ID NO: 11 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 107 aa, Homo sapiens [sequence omitted]

SEQ ID NO: 13 - DNA, 357 bp, Homo sapiens
  source 1..357: mol_type = unassigned DNA
caagttcagctggttcagtctggcgccgaggttgtgaaacctggcgcctctgtgaagatgagctgcaaggccagcggctacaccttcaccggctacaacatccactgggtcaagcaggcccctggacagggactcgaatggatcggagccatctatcccggcaacggcgacaccagctacaagcagaagttccggggcagagccacactgaccgccgatacaagcaccagcaccgtgtacatggaactgagcagcctgagaagcgaggacagcgccgtgtactattgcgccagaggcgaaacagccagagccacctttgcctattggggccagggaaccctggtcaccgttagctct

SEQ ID NO: 14 - DNA, 321 bp, Homo sapiens
  source 1..321: mol_type = unassigned DNA
gatattcagatgacacagagccccagcagcctgtctgcctctgtgggagacagagtgaccatcacctgtagcgccagccaggatatcggcaacttcctgaactggtatcagcagaaacccggcaagaccgtgaaggtgctgatctactacacctccagcctgtacagcggcgtgcccagcagattttctggcagcggctctggcaccgactacaccctgaccatatctagcctgcagcctgaggacttcgccacctactactgccagcagtacagcaagctgcccctgacatttggccagggcaccaagctggaactgaag

SEQ ID NO: 15 - protein, 690 aa, Homo sapiens
MAPWPELGDAQPNPDKYLEGAAGQQPTAPDKSKETNKTDNTEAPVTKIELLPSYSTATLIDEPTEVDDPWNLPTLQDSGIKWSERDTKGKILCFFQGIGRLILLLGFLYFFVCSLDILSSAFQLVGGKMAGQFFSNSSIMSNPLLGLVIGVLVTVLVQSSSTSTSIVVSMVSSSLLTVRAAIPIIMGANIGTSITNTIVALMQVGDRSEFRRAFAGATVHDFFNWLSVLVLLPVEVATHYLEIITQLIVESFHFKNGEDAPDLLKVITKPFTKLIVQLDKKVISQIAMNDEKAKNKSLVKIWCKTFTNKTQINVTVPSTANCTSPSLCWTDGIQNWTMKNVTYKENIAKCQHIFVNFHLPDLAVGTILLILSLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNALRSSLQIALCHFFFNISGILLWYPIPFTRLPIRMAKGLGNISAKYRWFAVFYLIIFFFLIPLTVFGLSLAGWRVLVGVGVPVVFIIILVLCLRLLQSRCPRVLPKKLQNWNFLPLWMRSLKPWDAVVSKFTGCFQMRCCCCCRVCCRACCLLCDCPKCCRCSKCCEDLEEAQEGQDVPVKAPETFDNITISREAQGEVPASDSKTECTAL